(19) United States
(12) Patent Application Publication
(10) Pub. No.: US 2025/0316339 A1
(43) Pub. Date: Oct. 9, 2025

(54) CANCER CLASSIFIER MODELS, MACHINE LEARNING SYSTEMS AND METHODS OF USE

(71) Applicants: Jonathan Cohen, Rockville, MD (US); Victoria Doseeva, Rockville, MD (US); Peichang Shi, Rockville, MD (US)

(72) Inventors: Jonathan Cohen, Rockville, MD (US); Victoria Doseeva, Rockville, MD (US); Peichang Shi, Rockville, MD (US)

(21) Appl. No.: 19/012,759

(22) Filed: Jan. 7, 2025

Related U.S. Application Data
(63) Continuation of application No. 16/458,589, filed on Jul. 1, 2019, now abandoned.
(60) Provisional application No. 62/692,683, filed on Jun. 30, 2018.

(51) Int. Cl. G16B 40/20; G06N 20/00; G16H 50/20; G16H 50/30

(52) U.S. Cl. CPC G16B 40/20; G06N 20/00; G16H 50/20; G16H 50/30

(57) ABSTRACT

Disclosed herein are classifier models, computer implemented systems, machine learning systems and methods thereof for classifying asymptomatic patients into a risk category for having or developing cancer and/or classifying a patient with an increased risk of having or developing cancer into an organ system-based malignancy class membership and/or into a specific cancer class membership.

### CROSS-REFERENCE TO RELATED APPLICATIONS

This application claims the benefit of U.S. Provisional Patent Application No. 62/692,683, filed on 30 Jun. 2018, the content of which is incorporated herein by reference in its entirety.

### FIELD OF THE INVENTION

This application pertains generally to classifier models generated by a machine learning system, trained with longitudinal data, for identifying asymptomatic patients with an increased risk for developing cancer and the type of cancer, especially in an otherwise asymptomatic or vaguely symptomatic patient.

### BACKGROUND OF THE INVENTION

For many types of cancers, patient outcomes improve significantly if surgery and other therapeutic interventions commence before the tumor has metastasized. Accordingly, imaging and diagnostic tests have been introduced into medical practice in an attempt to help physicians detect cancer early. These include various imaging modalities such as mammography as well as diagnostic tests to identify cancer specific “biomarkers” in the blood and other bodily fluids such as the prostate specific antigen (PSA) test. The value of many of these tests is often questioned particularly with regard to whether the costs and risks associated with false positives, false negatives, etc. outweigh the potential benefits in terms of actual lives saved. Furthermore, in order to demonstrate this value, data from large numbers of patients-many thousands or even tens of thousands—must be generated in real world (prospective) studies rather than retrospective analysis of laboratory stored samples. Unfortunately, the costs of conducting large prospective studies for screening tools is outweighed by reasonably anticipated financial returns so these large prospective studies are almost never done by the private sector and are only occasionally sponsored by governments. As a result, the use paradigms for blood testing for the early detection of most cancers has progressed little in several decades. In the United States, for example, PSA remains the only widely utilized blood test for cancer screening and even its utilization has become controversial. In other parts of the world, especially the Far East, blood tests for detecting various cancers is more commonplace but there is little standardization or empirical methods to ascertain or improve the accuracy of such testing in those parts of the world.

It would therefore be desirable to improve the accuracy and standardization of cancer screening in those regions where it is common and, in so doing, generate tools and technologies that may improve and/or encourage cancer screening in those regions where it is less common.

Cancer detection poses significant technical challenges as compared to detecting viral or bacterial infections since cancer cells, unlike viruses and bacteria, are biologically similar to and hard to distinguish from normal, healthy cells. For this reason, tests used for the early detection of cancer often suffer from higher numbers of false positives and false negatives than comparable tests for viral or bacterial infections or for tests that measure genetic, enzymatic, or hormonal abnormalities. This often causes confusion among healthcare practitioners and their patients leading in some cases to unnecessary, expensive, and invasive follow-up testing while in other cases to a complete disregard for follow-up testing resulting in cancers being detected too late for useful intervention. Physicians and patients welcome tests that yield a binary decision or result, e.g., either the patient is positive or negative for a condition, such as observed in the over the counter pregnancy test kits which present, for example, an immunoassay result in the shape of a plus sign or a negative sign as an indication of pregnancy or not. However, unless the sensitivity and specificity of diagnosis approaches 99%, a level not obtainable for most cancer tests, such binary outputs can be highly misleading or inaccurate.

It would therefore be desirable to provide healthcare practitioners and their patients with more quantitative information about their likelihood of having or developing cancer, and especially a particular cancer, even if a binary output is not practical.

Detecting early stage cancer is also challenging due to factors associated with the modern-day practice of medicine. Primary care providers in particular, see a high volume of patients per day and the demands of healthcare cost containment has dramatically shortened the amount of time they can spend with each patient. Accordingly, physicians often lack sufficient time to take in depth family and lifestyle histories, to counsel patients on healthy lifestyles, or to follow-up with patients who have been recommended testing beyond that which is provided in their office practice.

It would therefore be desirable to provide high-volume primary care providers, in particular, with useful tools to help them triage or compare the relative risks for their patients of having cancer so they can order additional testing for those patients at the highest risks.

Artificial intelligence/machine learning systems are useful for analyzing information and may assist human experts in decision making. For example, machine learning systems comprising diagnostic decision-support systems may use clinical decision formulas, rules, trees, or other processes for assisting a physician with making a diagnosis.

Although decision-making systems have been developed, such systems are not widely used in medical practice because these systems suffer from limitations that prevent them from being integrated into the day-to-day operations of health organizations. For example, decision-making systems may provide an unmanageable volume of data, rely on analysis that is marginally significant, and not correlate well with complex multimorbidity (Greenhalgh, T. Evidence based medicine: a movement in crisis? BMJ (2014) 348: g3725)

Many different healthcare workers may see a patient, and patient data may be scattered across different computer systems in both structured and unstructured form. Also, the systems are difficult to interact with (Berner, 2006; Shortliffe, 2006). The entry of patient data is difficult, the list of diagnostic suggestions may be too long, and the reasoning behind diagnostic suggestions is not always transparent. Further, the systems are not focused enough on next actions, and do not help the clinician figure out what to do to help the patient (Shortliffe, 2006).

It would, therefore, be desirable to provide methods and technologies to permit artificial intelligence/machine learning systems to be used to aid in the early detection of cancer, especially with blood testing.

### SUMMARY OF THE INVENTION

Disclosed herein are classifier models, machine learning systems, computer implemented systems and methods thereof.

In embodiments, a method, in a computer-implemented system comprising at least one processor and at least one memory, the at least one memory comprising instructions executed by the at least one processor to cause the at least one processor to implement one or more classifier models to predict an increased risk of having or developing cancer, for an asymptomatic patient, comprises obtaining measured values of a panel of biomarkers in a sample from the patient, wherein a value of a biomarker corresponds to a level of the biomarker in the sample; obtaining clinical parameters corresponding to the patient including at least age and gender; classifying the patient into a risk category of having or developing cancer using a first classifier model, wherein the first classifier model is generated by a machine learning system using first training data that comprises values of a panel of at least two biomarkers, age, and a diagnostic indicator, for a population of patients; and, wherein the first classifier model classifies the patient in an increased risk category using input variables of age and the measured values of a panel of biomarkers from the patient when an output of the first classifier model is above a threshold; and, providing a notification to a user for diagnostic testing of the patient when the patient is classified in the increased risk category.

In embodiments, the machine learning system further comprises iteratively regenerating the first classifier model by training the first classifier model with new training data to improve the performance of the first classifier model. In certain embodiments, the classifier model is iteratively regenerated wherein the method further comprises obtaining one or more test results from the diagnostic testing which confirm or deny the presence of cancer in the patient; incorporating the one or more test results into the first training data for further training of the first classifier model of the machine learning system; and generating an improved first classifier model by the machine learning system.

In certain embodiments, the training data used to train the classifier model generated by the machine learning system, comprises a group of data from a group of patients with no cancer diagnosis three or more months after providing a sample. In certain other embodiments, the training data comprises a group of data from a group of patients with a cancer diagnosis three or more months after providing a sample.

In other embodiments, a method, in a computer implemented system comprising at least one processor and at least one memory, the at least one memory comprising instructions executed by the at least one processor to cause the at least one processor to implement one or more classifier models to predict an organ system-based malignancy for a patient with an increased risk of having or developing cancer, comprises:
a) obtaining measured values of a panel of biomarkers in a sample from the patient, wherein a value of a biomarker corresponds to a level of the biomarker in the sample;
b) obtaining clinical parameters from the patient including at least age and gender;
c) classifying the patient into an organ system class membership using a cancer classifier model, wherein the cancer classifier model is generated by a machine learning system using training data that comprises values from a panel of at least two biomarkers, age, and a diagnostic indicator for a population of patients; and, wherein the cancer classifier model assigns the organ system class membership using input variables of age and the measured values of the panel of biomarkers from the patient; and,
d) providing a notification to a user for diagnostic testing of the patient when the patient is predicted to have the organ system-based malignancy.

In certain embodiments, provided herein is a method, in a computer implemented system comprising at least one processor and at least one memory, the at least one memory comprising instructions executed by the at least one processor to cause the at least one processor to implement one or more classifier models to predict an organ system-based malignancy for a patient with an increased risk of having or developing cancer, comprising:
a) obtaining measured values of a panel of biomarkers in a sample from the patient, wherein a value of a biomarker corresponds to a level of the biomarker in the sample;
b) obtaining clinical parameters corresponding to the patient including at least age and gender;
c) classifying the patient into a risk category of having or developing cancer using a first classifier model, wherein the first classifier model is generated by a machine learning system using first training data that comprises values of a panel of at least two biomarkers, age, and a diagnostic indicator, for a population of patients; and, wherein the first classifier model classifies the patient in an increased risk category using input variables of age and the measured values of a panel of biomarkers from the patient when an output of the first classifier model is above a threshold;
d) classifying the patient into an organ system class membership using a second classifier model, wherein the second classifier model is generated by a machine learning system Fusing training data that comprises values from a panel of at least two biomarkers, age, and a diagnostic indicator for a population of patients; and, wherein the cancer classifier model assigns the organ system class membership using input variables of age and the measured values of the panel of biomarkers from the patient; and,
e) providing a notification to a user for diagnostic testing of the patient when the patient is predicted to have the organ system-based malignancy.

In embodiments provided herein is a machine learning comprising at least one processor for predicting an organ system-based malignancy for a patient with an increased risk of having or developing cancer, wherein the processor is configured to:
a) obtain measured values of a panel of biomarkers in a sample from the patient, wherein a value of a biomarker corresponds to a level of the biomarker in the sample;
b) obtain clinical parameters from the patient including age and gender;
c) generate a first classifier model by the machine learning system to classify the patient into a risk category of having or developing cancer, wherein the first classifier model classifies a patient into an increased risk category when the output of the first classifier model is greater than a threshold, and wherein the first classifier model is generated by the machine learning system using training data that comprises values from a panel of at least six biomarkers, age, gender and a diagnostic indicator for a population of patients;
d) generate a second classifier model by the machine learning system to classify the patient into an organ system class membership,
   wherein the cancer classifier model assigns the organ system class membership using input variables of age and the measured values of the panel of biomarkers from the patient, and
   wherein the second classifier model is generated by a machine learning system using training data that comprises values from a panel of at least two biomarkers, age, and a diagnostic indicator for a population of patients; and,
e) provide a notification to a user for diagnostic testing of the patient.

### BRIEF DESCRIPTION OF THE DRAWINGS

The drawings illustrate generally, by way of example, but not by way of limitation, various embodiments disclosed herein.

FIGS. 1A and 1B show Receiver Operating Characteristic (ROC) Curves for the best performing machine learning models, Ridge Logistic Regression (AUC 0.875, Youden Index 0.628) (FIG. 1A) and SVM model (AUC 0.816, Youden Index 0.631) (FIG. 1B) for male subject's likelihood of developing cancer within about 2 years from testing date. See Example 1 and Table 4.

FIG. 2 shows the performance of pattern recognition algorithm (kNN) for determining the top three (N=3) organ systems from individuals classified as “Moderate Risk” or “High Risk” for developing cancer. The x-axes represents the number of organ systems, wherein the “top 3” is highlighted and the y-axes represents the percentage (%) of either the sensitivity or specificity as indicated by the overlapping dashed lines for “sensitivity” and “specificity”. This algorithm was trained to predict organ system-based malignancy risk in individuals with a probability greater than 0.5 for developing pan-cancer. See Example 2.

FIG. 3 shows a table of input variables (biomarker measurements and age) for the classifier model and the classification of each patient into a risk category based on the output (probability value). See Example 3.

FIG. 4 shows the workflow for performing methods to predict an increased risk of having or developing cancer, for an asymptomatic patient using the present classifier models.

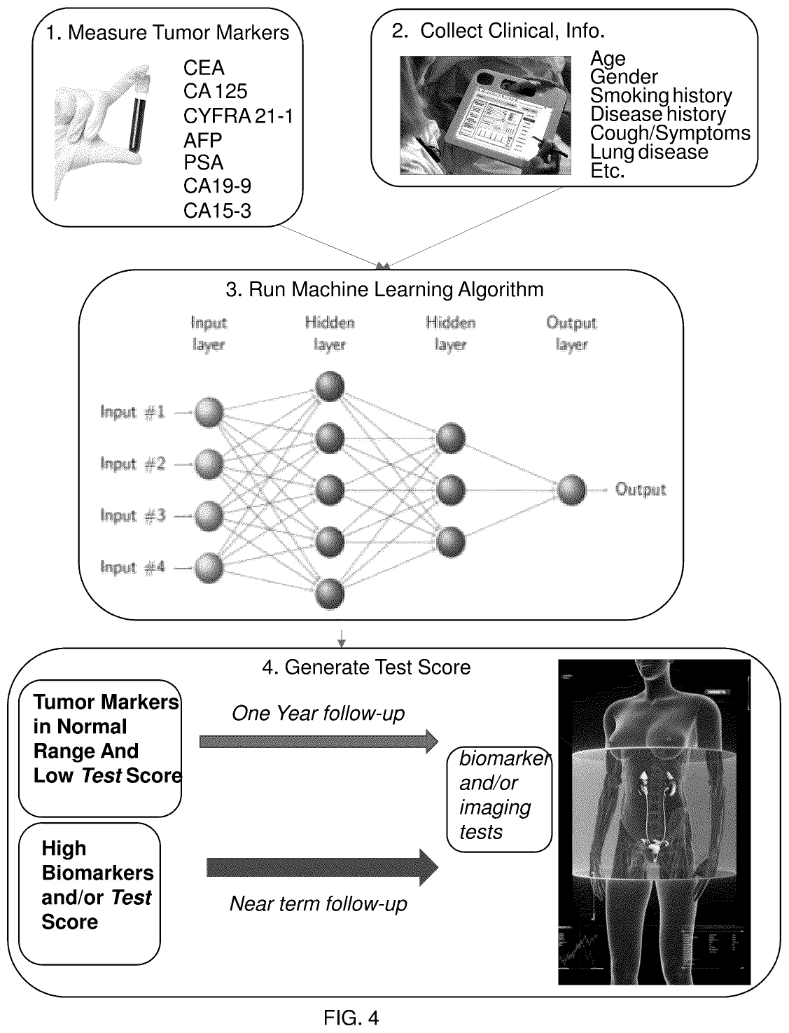

FIGS. 5A and 5B show the present male classifier model is significantly better at predicting cancer development within one year than measurement of a panel of individual biomarkers from the same subjects (FIG. 5A) and the corresponding ROC (Receiver Operator Curve) indicating area under the curve (AUC) value of 0.87 for a population of male patients using the present male classifier model “AUC of Male” (FIG. 5B). See Example 4. The table of FIG. 5A includes a header of “Model” indicating a present algorithm (e.g., male classifier model) was used (“yes”) based on the header of “Algorithm”, or the “Single threshold method”, which does not (“none”) include an algorithm to be used, a measured value of the biomarker is comparted to a threshold value “any marker high”. The present male classifier model is an improvement as compared to individual biomarker “single threshold” method wherein the sensitivity represents a 2-fold increase as compared to the single threshold method. In other words, the present male classifier model identifies 2× more cancers in male patients as compared to the conventional methods of “any marker high”.

FIGS. 6A and 6B show the present male classifier model was able to distinguish cancers from noncancers with 82% sensitivity and 81% specificity with a threshold value of 0.5.

FIGS. 7A and 7B show the present female classifier model is significantly better at predicting cancer development within one year than measurement of a panel of individual biomarkers from the same subjects (FIG. 7A) and the ROC (Receiver Operator Curve) indicating the AUC value of 0.67 for a population of female patients using the present female classifier model “AUC of Female” (FIG. 7B). The present female classifier model is an improvement as compared to individual biomarker “single threshold” method wherein the sensitivity represents a 4-fold increase as compared to the single threshold method. In other words, the present female classifier model identifies 4× more cancers in female patients as compared to the conventional methods of “any marker high”.

FIGS. 8A and 8B show the present female classifier model was able to distinguish cancers from noncancers with 50% sensitivity and 74% specificity with a threshold value of 0.5.

### DETAILED DESCRIPTION OF THE INVENTION

Embodiments of the present invention relate generally to non-invasive methods, diagnostic tests, especially blood (including serum or plasma) tests that measure biomarkers (e.g. tumor antigens) in combination with clinical parameters, and classification models generated by a machine learning system, assigning a patient to a risk category for having or developing cancer, and assigning a patient classified into an increased risk category for having or developing cancer, to an organ system class membership to determine whether that patient should be followed up with additional, more invasive diagnostic testing.

### Introduction

Disclosed herein are classifier models and their use with patients asymptomatic for cancer for the early prediction of tumors and/or occult cancer. The classifier models were generated by a machine learning system using training data that comprises values of a panel of at least two biomarkers, age, and a diagnostic indicator, for a population of patients. The present classifier models were trained with biomarkers that were measured at least 3 months, if not longer, before patients received a diagnosis. In embodiments, training data comprises a group of data from a group of patients with no cancer diagnosis three or more months after providing a sample. In embodiments, the training data comprises a group of data from a group of patients with a cancer diagnosis three or more months after providing a sample. See Example 1A.

In the present invention, the classifier models are “trained” using machine learning systems by building a model from inputs. Those inputs may be longitudinal data, wherein a known diagnosis of cancer (including matched controls) is determine months, if not years after data from measured biomarkers and clinical factors of those patients is collected. See Example 1A and 2 for training of the present classifier models using longitudinal cancer patient data.

Provided herein is a first classifier model generated by a machine learning system wherein inclusion of age as an input variable (along with a panel of biomarker values), and for training of the model, significantly, and unexpectedly, increased the performance of the first classifier model. See Example 1B. In embodiments, the classifier model has a performance of a Receiver Operator Characteristic (ROC) curve with a sensitivity value of at least 0.8 and a specificity value of at least 0.8 for correct classification of the patient as having or developing cancer.

In embodiments provided herein is a first classifier model, generated by a machine learning system, that classifies a patient into a risk category of having or developing cancer. In embodiments, use of the classifier model classifies a patient in an increased risk category using input variables of age and the measured values of a panel of biomarkers from the patient when an output of the classifier model is above a threshold value. In other embodiments, the classifier model classifies a patient in a low risk category using input variables of age and the measured values of a panel of biomarkers from the patient when an output of the classifier model is below a threshold value. As used herein, the term “increased risk” refers to an increase for the presence, or development, of the cancer as compared to the known prevalence of that particular cancer across the population cohort. See Example 3.

In embodiments provided herein is a second classifier model, generated by a machine learning system, that classifies a patient into an organ system or specific cancer class membership. In embodiments, the second classifier model assigns the organ system or specific cancer class membership using input variables of age and the measured values of the panel of biomarkers from the patient. In certain embodiments, a patient is classified into an organ system or specific cancer class membership using a second classifier model, when the patient was classified into an increased risk category by the first classifier model, and wherein the second classifier model is generated by a machine learning system using training data that comprises values from a panel of at least two biomarkers, age, and a diagnostic indicator for a population of patients.

In certain embodiments the classifier model is static, and its use is implemented by a computer-implemented system comprising at least one processor and at least one memory, the at least one memory comprising instructions executed by the at least one processor to cause the at least one processor to implement the classifier model. In certain embodiments, a machine learning system iteratively regenerates the classifier model by training the classifier model with new training data to improve the performance of the classifier model.

In exemplary embodiments, the present methods using a first classifier model, and in a computer-implemented system comprising at least one processor and at least one memory, the at least one memory comprising instructions executed by the at least one processor to cause the at least one processor to implement one or more classifier models to predict an increased risk of having or developing cancer, for an asymptomatic patient, comprising obtaining measured values of a panel of biomarkers in a sample from the patient, wherein a value of a biomarker corresponds to a level of the biomarker in the sample, obtaining clinical parameters corresponding to the patient including at least age and gender, classifying the patient into a risk category of having or developing cancer using a first classifier model, wherein the first classifier model is generated by a machine learning system using first training data that comprises values of a panel of at least two biomarkers, age, and a diagnostic indicator, for a population of patients; and, wherein the first classifier model classifies the patient in an increased risk category using input variables of age and the measured values of a panel of biomarkers from the patient when an output of the first classifier model is above a threshold and providing a notification to a user for diagnostic testing of the patient when the patient is classified in the increased risk category. See Example 1 and 3.

In other exemplary embodiments, the present methods using a second classifier model, and in a computer implemented system comprising at least one processor and at least one memory, the at least one memory comprising instructions executed by the at least one processor to cause the at least one processor to implement one or more classifier models to predict an organ system-based malignancy for a patient with an increased risk of having or developing cancer, comprise obtaining measured values of a panel of biomarkers in a sample from the patient, wherein a value of a biomarker corresponds to a level of the biomarker in the sample, obtaining clinical parameters from the patient including at least age and gender, classifying the patient into an organ system class membership using a second classifier model, wherein the classifier model is generated by a machine learning system using training data that comprises values from a panel of at least two biomarkers, age, and a diagnostic indicator for a population of patients; and, wherein the cancer classifier model assigns the organ system class membership using input variables of age and the measured values of the panel of biomarkers from the patient; and, providing a notification to a user for diagnostic testing of the patient when the patient is predicted to have the organ system-based malignancy. See Example 2 and 3.

The first classifier model yields a numerical risk score for each patient tested, which can be used by physicians to further inform screening procedures to better predict and diagnose early stage cancer in asymptomatic patients. Those patients classified into an increased risk category may be further classified using the second classifier model into a class membership. That class membership may be an organ system malignancy, or a specific cancer type. Also, as disclosed in more detail herein, the machine learning system is adapted to receive additional data as the system is used in a real-world clinical setting and to recalculate and improve the performance so that the classifier model becomes “smarter” the more it is used.

### Definitions

As used herein, the terms “a” or “an” are used, as is common in patent documents, to include one or more than one, independent of any other instances or usages of “at least one” or “one or more.”

As used herein, the term “or” is used to refer to a nonexclusive or, such that “A or B” includes “A but not B,” “B but not A,” and “A and B,” unless otherwise indicated.

As used herein, the term “about” is used to refer to an amount that is approximately, nearly, almost, or in the vicinity of being equal to or is equal to a stated amount, e.g., the state amount plus/minus about 5%, about 4%, about 3%, about 2% or about 1%.

As used herein, the term “asymptomatic” refers to a patient or human subject that has not previously been diagnosed with the same cancer that their risk of having is now being quantified and categorized. For example, human subjects may show signs such as coughing, fatigue, pain, etc., but have not been previously diagnosed with lung cancer but are now undergoing screening to categorize their increased risk for the presence of cancer and for the present methods are still considered “asymptomatic”.

As used herein, the term “AUC” refers to the Area Under the Curve, for example, of a ROC Curve. That value can assess the merit or performance of a test on a given sample population with a value of 1 representing a good test ranging down to 0.5 which means the test is providing a random response in classifying test subjects. Since the range of the AUC is only 0.5 to 1.0, a small change in AUC has greater significance than a similar change in a metric that ranges for 0 to 1 or 0 to 100%. When the % change in the AUC is given, it will be calculated based on the fact that the full range of the metric is 0.5 to 1.0. A variety of statistics packages can calculate AUC for a ROC curve, such as, JMP™ or Analyse-It™. AUC can be used to compare the accuracy of the classification model across the complete data range. Classification models with greater AUC have, by definition, a greater capacity to classify unknowns correctly between the two groups of interest (disease and no disease).

As used herein, the terms “biological sample” and “test sample” refer to all biological fluids and excretions isolated from any given subject. In the context of embodiments of the present invention such samples include, but are not limited to, blood, blood serum, blood plasma, urine, tears, saliva, sweat, biopsy, ascites, cerebrospinal fluid, milk, lymph, bronchial and other lavage samples, or tissue extract samples. In certain embodiments, blood, serum, plasma and bronchial lavage or other liquid samples are convenient test samples for use in the context of the present methods.

As used herein, a “biomarker measure” is information relating to a biomarker that is useful for characterizing the presence or absence of a disease. Such information may include measured values which are, or are proportional to, concentration, or that are otherwise provide qualitative or quantitative indications of expression of the biomarker in tissues or biologic fluids.

As used herein, the terms “cancer” and “cancerous” refer to or describe the physiological condition in mammals that is typically characterized by unregulated cell growth.

Examples of cancer include but are not limited to, lung cancer, breast cancer, colon cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, and brain cancer.

As used herein, the term “cohort” or “cohort population” refers to a group or segment of human subjects with shared factors or influences, such as age, family history, cancer risk factors, environmental influences, medical histories, etc. In one instance, as used herein, a “cohort” refers to a group of human subjects with shared cancer risk factors; this is also referred to herein as a “disease cohort”. In another instance, as used herein, a “cohort” refers to a normal population group matched, for example by age, to the cancer risk cohort; also referred to herein as a “normal cohort”. A “same cohort” refers to a group of human subjects having the same shared cancer risk factors as the individual undergoing assessment for a risk of having a disease such as cancer.

As used herein “machine learning” refers to algorithms that give a computer the ability to learn without being explicitly programmed including algorithms that learn from and make predictions about data. Machine learning algorithms include, but are not limited to, decision tree learning, artificial neural networks (ANN) (also referred to herein as a “neural net”), deep learning neural network, support vector machines, rule base machine learning, random forest, logistic regression, pattern recognition algorithms, etc. For the purposes of clarity, algorithms such as linear regression or logistic regression can be used as part of a machine learning process. However, it is understood that using linear regression or another algorithm as part of a machine learning process is distinct from performing a statistical analysis such as regression with a spreadsheet program such as Excel. The machine learning process has the ability to continually learn and adjust the classifier model as new data becomes available and does not rely on explicit or rules-based programming. Statistical modeling relies on finding relationships between variables (e.g., mathematical equations) to predict an outcome.

As used herein, the term “medical history” refers to any type of medical information associated with a patient. In some embodiments, the medical history is stored in an electronic medical records database. Medical history may include clinical data (e.g., imaging modalities, blood work, biomarkers, cancerous samples and control samples, labs, etc.), clinical notes, symptoms, severity of symptoms, number of years smoking, family history of a disease, history of illness, treatment and outcomes, an ICD code indicating a particular diagnosis, history of other diseases, radiology reports, imaging studies, reports, medical histories, genetic risk factors identified from genetic testing, genetic mutations, etc.

As used herein, the term “increased risk” refers to an increase in the risk level, for a human subject after analysis by the classifier model, for the presence, or development, of a cancer relative to a population's known prevalence of a particular cancer before testing. In other words, a human subject's risk for cancer before biomarker testing and/or data analysis may be 1% (based on the understood prevalence of cancer in the population), but after analysis using the classifier model the patient's risk for the presence of cancer may be 8% or alternatively reported as an increase of 8 times compared to the cohort. The machine learning system calculates the 8% risk of having the cancer and the increased risk of 8 times relative to the population or cohort population is provided in more detail herein.

As used herein, the terms “marker”, “biomarker” (or fragment thereof) and their synonyms, which are used interchangeably, refer to molecules that can be evaluated in a sample and are associated with a physical condition. For example, markers include expressed genes or their products (e.g., proteins) or autoantibodies to those proteins that can be detected from human samples, such as blood, serum, solid tissue, and the like, that is associated with a physical or disease condition. Such biomarkers include, but are not limited to, biomolecules comprising nucleotides, amino acids, sugars, fatty acids, steroids, metabolites, polypeptides, proteins (such as, but not limited to, antigens and antibodies), carbohydrates, lipids, hormones, antibodies, regions of interest which serve as surrogates for biological molecules, combinations thereof (e.g., glycoproteins, ribonucleoproteins, lipoproteins) and any complexes involving any such biomolecules, such as, but not limited to, a complex formed between an antigen and an autoantibody that binds to an available epitope on said antigen. The term “biomarker” can also refer to a portion of a polypeptide (parent) sequence that comprises at least 5 consecutive amino acid residues, preferably at least 10 consecutive amino acid residues, more preferably at least 15 consecutive amino acid residues, and retains a biological activity and/or some functional characteristics of the parent polypeptide, e.g. antigenicity or structural domain characteristics. The present markers refer to both tumor antigens present on or in cancerous cells or those that have been shed from the cancerous cells into bodily fluids such as blood or serum. The present markers, as used herein, also refer to autoantibodies produced by the body to those tumor antigens. In one aspect, a “marker” as used herein refers to both tumor antigens and autoantibodies that are capable of being detected in serum of a human subject. It is also understood in the present methods that use of the markers in a panel may each contribute equally in the classifier model or certain biomarkers may be weighted wherein the markers in a panel contribute a different weight or amount in the classifier model. Biomarker may include any biological substance indicative of the presence of cancer, including but not limited to, genetic, epigenetic, proteomic, glycomic or imaging biomarkers. Biomarkers include molecules secreted by tumors or cancer, including cell freeDNA, RNA, and protein-based products (tumor markers or antigens), etc.

As used herein, the term “pathology” of (tumor) cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, a “physiological sample” includes samples from biological fluids and tissues. Biological fluids include whole blood, blood plasma, blood serum, sputum, urine, sweat, lymph, and alveolar lavage. Tissue samples include biopsies from solid lung tissue or other solid tissues, lymph node biopsy tissues, biopsies of metastatic foci. Methods of obtaining physiological samples are well known.

As used herein, the term “a positive predictive score,” “a positive predictive value,” or “PPV” refers to the likelihood that a score within a certain range on a biomarker test is a true positive result. It is defined as the number of true positive results divided by the number of total positive results. True positive results can be calculated by multiplying the test sensitivity times the prevalence of disease in the test population. False positives can be calculated by multiplying (1 minus the specificity) times (1−the prevalence of disease in the test population). Total positive results equal True Positives plus False Positives.

As used herein the term, “Receiver Operating Characteristic Curve,” or, “ROC curve,” is a plot of the performance of a particular feature for distinguishing two populations, patients with cancer, and controls, e.g., those without cancer. Data across the entire population (namely, the patients and controls) are sorted in ascending order based on the value of a single feature. Then, for each value for that feature, the true positive and false positive rates for the data are determined. The true positive rate is determined by counting the number of cases above the value for that feature under consideration and then dividing by the total number of patients. The false positive rate is determined by counting the number of controls above the value for that feature under consideration and then dividing by the total number of controls.

ROC curves can be generated for a single feature as well as for other single outputs, for example, a combination of two or more features that are combined (such as, added, subtracted, multiplied, weighted, etc.) to provide a single combined value which can be plotted in a ROC curve. The ROC curve is a plot of the true positive rate (sensitivity) of a test against the false positive rate (1-specificity) of the test. ROC curves provide another means to quickly screen a data set. As used herein, performance of the present classifier models is determined using computed ROC curves with sensitivity and specificity values. The performance is used to compare models, and also importantly, to compare models with different variables to select a classifier model with the highest accuracy as to predicting having or developing cancer, for a patient.

Classifier Models Generated by Machine Learning Systems and their Use

Disclosed herein are classifier models, computer implemented systems, machine learning systems and methods thereof for classifying asymptomatic patients into a risk category for having or developing cancer and/or classifying a patient with an increased risk of having or developing cancer into an organ system-based malignancy class membership and/or into a specific cancer class membership.

The machine learning system disclosed herein generated the present classifier models using longitudinal data from a cohort of over 12,000 asymptomatic male patients and over 15,000 asymptomatic female patients. See Example 1A and 2. In this instance biomarkers were measured, and follow-up of the patients was performed to provide a diagnostic indicator in the future (e.g. no cancer development, or diagnosis of a specific cancer). Using biomarkers obtained months, or even years, before cancer was detected provided a powerful tool to train the classifier models resulting in highly accurate classifier models as measured by ROC curve analysis. In embodiments, training data comprises data from a group of patients with no cancer diagnosis three or more months after providing a sample. In embodiments, training data comprises data from a group of patients with a cancer diagnosis three or more months after providing a sample.

In embodiments, the cohort of asymptomatic female patients was used to train a classifier model to be used with female patients and the cohort of asymptomatic male patients was used to train a classifier model to be used with male patients. In embodiments, the gender of the patient is used to select the classifier model. In embodiments, training data comprises a greater number of patients without cancer than with cancer, wherein training of the classifier models comprises reprocessing the training data by using a stratified sampling technique to improve selection of negative samples.

Surprisingly, including age as an input variable for training and use of the classifier model further improved the performance of the classifier models. See Example 1B. In embodiments, the classifier model has a performance of a Receiver Operator Characteristic (ROC) curve with a sensitivity value of at least 0.8 and a specificity value of at least 0.8 for correct classification of the patient as having or developing cancer.

In embodiments, the machine learning system generates a classifier model that may be static. In other words, the classifier model is trained and then its use is implemented with a computer implemented system wherein patient data (e.g. biomarker marker measurements and age) are input and the classifier model provides an output that is used to classify patients.

In other embodiments, the classifier models are continuously, or routinely, being updated and improved wherein the input values, output values, along with a diagnostic indicator from patients are used to further train the classifier models. In embodiments, the classifier model has an improved performance of a Receiver Operator Characteristic (ROC) curve having a sensitivity value of at least 0.85 and a specificity value of at least 0.8.

In embodiments, the classifier model is further trained and improved by the machine learning system comprising (1) obtaining one or more test results from the diagnostic testing which confirm or deny the presence of cancer in the patient, (2) incorporating the one or more test results into the training data for further training of the classifier model of the machine learning system; and (3) generating an improved classifier model by the machine learning system. In embodiments, diagnostic testing comprises radiography screening or tissue biopsy.

In embodiments provided herein is a classifier model to predict an increased risk of having or developing cancer, for an asymptomatic patient. In embodiments, this first classifier model is generated by a machine learning system using training data that comprises values of a panel of at least two biomarkers, age, and a diagnostic indicator, for a population of patients. In embodiments, the first classifier model was trained using data from only a male cohort or a female cohort. In embodiments, the training data that comprises values of a panel of at least six biomarkers. In embodiments, the training data comprises values from a panel of biomarkers selected from AFP, CEA, CA125, CA19-9, CA 15-3, CYFRA21-1, PSA and SCC.

In exemplary embodiments, a first classifier model is generated by a machine learning system using training data that comprises a male cohort only, values of a panel of six biomarkers comprising AFP, CEA, CA19-9, CYFRA21-1, PSA and SCC, and age. In other exemplary embodiments, a first classifier model is generated by a machine learning system using training data that comprises a female cohort only, values of a panel of seven biomarkers comprising AFP, CEA, CA125, CA19-9, CA 15-3, CYFRA21-1 and SCC, and age.

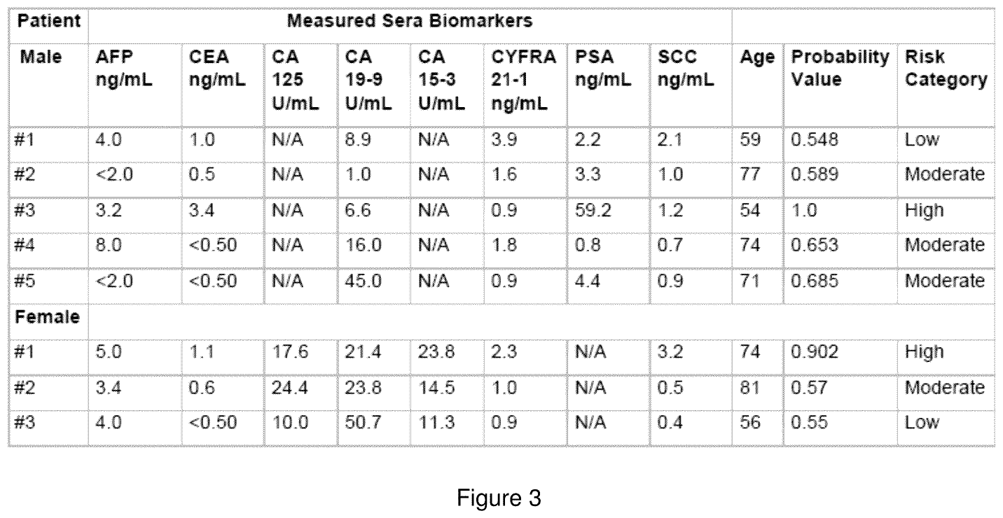

In embodiments, the first classifier model classifies the patient in an increased risk category using input variables of age and the measured values of a panel of biomarkers from the patient when an output of the first classifier model is above a threshold. In embodiments, the first classifier model classifies the patient in a low (e.g., no increased risk) risk category using input variables of age and the measured values of a panel of biomarkers from the patient when an output of the first classifier model is below a threshold. In exemplary embodiments, the output is a probability value, wherein the threshold is set to separate patients into a low risk category (those patients wherein their risk is no more than the population reflective of the training data) from an increased risk category (those patients with an increased risk of having or developing cancer as compared to a population reflective of the training data). See Example 3 and FIG. 3. In certain embodiments, the increased risk category may be further subdivided, such as a moderate risk category and a high-risk category.

In embodiments, those patients classified into an increased risk category may be assigned a risk score, such as a percent, e.g., X of 100, or multiplier number. In certain embodiments, a patient may be assigned a 2 to 10% risk score (of having or developing cancer) wherein the incidence of cancer in the population used to train the classifier model is about 1%. In embodiments, those percentage risk scores may be presented as X of 100, e.g. 3 out of 100 wherein a patient with that score has an approximately 3 out of 100 risk of developing cancer within one year from when the biomarkers were measured. In this instance, a threshold cut off, wherein a risk score at or below would be considered normal, and a risk score above would be considered an increased risk. In certain embodiments, the threshold cut off value may be 1 out of 100, corresponding to a “normal” risk of having cancer in a heterogenous population of 1%.

In certain other embodiments, the patient may be assigned a multiplier number. In embodiments, the risk score is not an output value, but a value assigned to a risk category, such as an increased risk category, wherein the output value is used to classify a patient into the risk category. In certain embodiments, an output value is a predicted probability value that may range from 0 to 1, wherein that value is used to classify a patient into a risk category. The risk score assigned to a risk category is then calculated by comparing the predicted probability assigned to a risk category to the prevalence of cancer in a population. See Example 3.

In embodiments, a patient may have an increased risk of having or developing cancer selected from the group consisting of: breast cancer, bile duct cancer, bone cancer, cervical cancer, colon cancer, colorectal cancer, gallbladder cancer, kidney cancer, liver or hepatocellular cancer, lobular carcinoma, lung cancer, melanoma, ovarian cancer, pancreatic cancer, prostate cancer, skin cancer, and testicular cancer.

In embodiments, the classifier model is selected based on the gender of the patient. In embodiments, the input variables for a male patient comprises measured values from a panel of at least six biomarkers and age. In embodiments, the panel of biomarkers is selected from AFP, CEA, CA125, CA19-9, CA 15-3, CYFRA21-1, PSA and SCC. In exemplary embodiments, the input variable for a male patient comprises measured values from AFP, CEA, CA19-9, CYFRA21-1, PSA and SCC, and age. In other embodiments, the input variables for a female patient comprises measured values from a panel of at least six biomarkers and age. In exemplary embodiments, the input variables for a female patent comprises measured values from AFP, CEA, CA125, CA19-9, CA 15-3, CYFRA21-1 and SCC, and age.

In embodiments, the first classifier model comprises a support vector machine, a decision tree, a random forest, a neural network, a deep learning neural network, or a logistic regression algorithm.

Disclosed herein is a second classifier model to predict at least one most likely organ system malignancy and/or a specific cancer. In certain embodiments, the second classifier model is applied to patients that are classified into an increased risk category for having or developing cancer. As with the first classifier model, the second classifier model was trained with measured biomarkers from a longitudinal study, and age, wherein one classifier model was trained from and for female patients and another classifier model was trained from and for male patients.

In embodiments, the second classifier model was generated by a machine learning system using training data that comprises values from a panel of at least two biomarkers, age, and a diagnostic indicator for a population of patients. In embodiments, the second classifier model was trained using data from only a male cohort or only a female cohort. In embodiments, the training data comprises values of a panel of at least six biomarkers. In embodiments, the training data comprises values from a panel of biomarkers selected from AFP, CEA, CA125, CA19-9, CA 15-3, CYFRA21-1, PSA and SCC.

In exemplary embodiments, a second classifier model is generated by a machine learning system using training data that comprises a male cohort only, values of a panel of six biomarkers comprising AFP, CEA, CA19-9, CYFRA21-1, PSA and SCC, and age. In other exemplary embodiments, a second classifier model is generated by a machine learning system using training data that comprises a female cohort only, values of a panel of seven biomarkers comprising AFP, CEA, CA125, CA19-9, CA 15-3, CYFRA21-1 and SCC, and age. In embodiments, the second classifier model has a performance of a Receiver Operator Characteristic (ROC) curve with a sensitivity value of at least 0.8 and a specificity value of at least 0.7 for correct classification of the patient as having or developing cancer.

In embodiments, the second classifier model assigns a patient into an organ system class membership using input variables of age and the measured values of the panel of biomarkers from the patient. In certain embodiments, the second classifier model assigns a patient into a specific cancer class membership using input variables of age and the measured values of the panel of biomarkers from the patient. In embodiments, the class membership is for an organ system selected from genitourinary (GU), gastrointestinal (GI), pulmonary, dermatological, hematological, nervous system, gynecological, or general. See Example 3. In certain embodiments, the class membership is for a cancer selected from breast cancer, bile duct cancer, bone cancer, cervical cancer, colon cancer, colorectal cancer, gallbladder cancer, kidney cancer, liver or hepatocellular cancer, lobular carcinoma, lung cancer, melanoma, ovarian cancer, pancreatic cancer, prostate cancer, skin cancer, or testicular cancer.

In embodiments, the second classifier model is selected based on the gender of the patient. In embodiments, the input variables for a male patient comprises measured values from a panel of at least six biomarkers and age. In embodiments, the panel of biomarkers is selected from AFP, CEA, CA125, CA19-9, CA 15-3, CYFRA21-1, PSA and SCC. In exemplary embodiments, the input variable for a male patient comprises measured values from AFP, CEA, CA19-9, CYFRA21-1, PSA and SCC, and age. In other embodiments, the input variables for a female patient comprises measured values from a panel of at least six biomarkers and age. In exemplary embodiments, the input variables for a female patent comprises measured values from AFP, CEA, CA125, CA19-9, CA 15-3, CYFRA21-1 and SCC, and age.

In embodiments, the second classifier model comprises a pattern recognition algorithm. In exemplary embodiments, the second classifier model comprises k-Nearest Neighbors algorithm (kNN). In certain embodiments, the second classifier model comprises a support vector machine, a decision tree, a random forest, a neural network, a deep learning neural network, or a logistic regression algorithm.

Disclosed herein is a machine learning system comprising at least one processor for predicting an increased risk for cancer, and/or an organ system-based malignancy, and/or a specific cancer.

In certain embodiments, the processor is configured to obtain measured values of a panel of biomarkers in a sample from a patient, wherein a value of a biomarker corresponds to a level of the biomarker in the sample, obtain clinical parameters from the patient including age and gender, and generate a first classifier model by the machine learning system to classify the patient into a risk category of having or developing cancer, wherein the first classifier model classifies a patient into an increased risk category when the output of the first classifier model is greater than a threshold, and wherein the first classifier model is generated by the machine learning system using training data that comprises values from a panel of at least two biomarkers, age, gender and a diagnostic indicator for a population of patients. In embodiments, the training data is from longitudinal study wherein the biomarker measurements are obtained months, or years, before a cancer diagnosis is confirmed (or not) for a patent in the training data cohort.

In certain other embodiments, the processor is configured to obtain measured values of a panel of biomarkers in a sample from the patient, wherein a value of a biomarker corresponds to a level of the biomarker in the sample; obtain clinical parameters from the patient including age and gender, and generate a second classifier model by the machine learning system to classify the patient into an organ system class membership, wherein the second classifier model assigns the organ system class membership using input variables of age and the measured values of the panel of biomarkers from the patient, and wherein the second classifier model is generated by a machine learning system using training data that comprises values from a panel of at least two biomarkers, age, and a diagnostic indicator for a population of patients.

In certain other embodiments, the processor is configured to obtain measured values of a panel of biomarkers in a sample from the patient, wherein a value of a biomarker corresponds to a level of the biomarker in the sample; obtain clinical parameters from the patient including age and gender, and generate a second classifier model by the machine learning system to classify the patient into a specific cancer class membership, wherein the second classifier model assigns the specific cancer class membership using input variables of age and the measured values of the panel of biomarkers from the patient, and wherein the second classifier model is generated by a machine learning system using training data that comprises values from a panel of at least two biomarkers, age, and a diagnostic indicator for a population of patients. Measuring Biomarkers in a Sample

As part of the present method, a panel of markers from an asymptomatic human subject may be measured. There are many methods known in the art for measuring either gene expression (e.g., mRNA) or the resulting gene products (e.g., polypeptides or proteins) that can be used in the present methods, and known to one of skill in the art. However, for at least 2-3 decades tumor antigens (e.g. CEA, CA-125, PSA, etc.) have been the most widely utilized biomarkers for cancer detection throughout the world and are the preferred tumor marker type for the present invention.

For tumor antigen detection, testing is preferably conducted using an automated immunoassay analyzer from a company with a large installed base. Representative analyzers include the Elecsys® system from Roche Diagnostics or the Architect® Analyzer from Abbott Diagnostics. Using such standardized platforms permits the results from one laboratory or hospital to be transferable to other laboratories around the world. However, the methods provided herein are not limited to any one assay format or to any particular set of markers that comprise a panel. For example, PCT International Pat. Pub. No. WO 2009/006323; US Pub. No. 2012/0071334; US Pat. Pub. No. 2008/0160546; US Pat. Pub. No. 2008/0133141; US Pat. Pub. No. 2007/0178504 (each herein incorporated by reference) teaches a multiplex lung cancer assay using beads as the solid phase and fluorescence or color as the reporter in an immunoassay format. Hence, the degree of fluorescence or color can be provided in the form of a qualitative score as compared to an actual quantitative value of reporter presence and amount.

For example, the presence and quantification of one or more antigens or antibodies in a test sample can be determined using one or more immunoassays that are known in the art. Immunoassays typically comprise: (a) providing an antibody (or antigen) that specifically binds to the biomarker (namely, an antigen or an antibody); (b) contacting a test sample with the antibody or antigen; and (c) detecting the presence of a complex of the antibody bound to the antigen in the test sample or a complex of the antigen bound to the antibody in the test sample.

Well known immunological binding assays include, for example, an enzyme linked immunosorbent assay (ELISA), which is also known as a “sandwich assay”, an enzyme immunoassay (EIA), a radioimmunoassay (RIA), a fluoroimmunoassay (FIA), a chemiluminescent immunoassay (CLIA), a counting immunoassay (CIA), a filter media enzyme immunoassay (META), a fluorescence-linked immunosorbent assay (FLISA), agglutination immunoassays and multiplex fluorescent immunoassays (such as the Luminex Lab MAP), immunohistochemistry, etc. For a review of the general immunoassays, see also, Methods in Cell Biology: Antibodies in Cell Biology, volume 37 (Asai, ed. 1993); Basic and Clinical Immunology (Daniel P. Stites; 1991).

The immunoassay can be used to determine a test amount of an antigen in a sample from a subject. First, a test amount of an antigen in a sample can be detected using the immunoassay methods described above. If an antigen is present in the sample, it will form an antibody-antigen complex with an antibody that specifically binds the antigen under suitable incubation conditions as described herein. The amount, activity, or concentration, etc. of an antibody-antigen complex can be determined by comparing the measured value to a standard or control. The AUC for the antigen can then be calculated using techniques known, such as, but not limited to, a ROC analysis.

In another embodiment, gene expression of markers (e.g., mRNA) is measured in a sample from a human subject. For example, gene expression profiling methods for use with paraffin-embedded tissue include quantitative reverse transcriptase polymerase chain reaction (qRT-PCR), however, other technology platforms, including mass spectroscopy and DNA microarrays can also be used. These methods include, but are not limited to, PCR, Microarrays, Serial Analysis of Gene Expression (SAGE), and Gene Expression Analysis by Massively Parallel Signature Sequencing (MPSS).

Any methodology that provides for the measurement of a marker or panel of markers from a human subject is contemplated for use with the present methods. In certain embodiments, the sample from the human subject is a tissue section such as from a biopsy. In another embodiment, the sample from the human subject is a bodily fluid such as blood, serum, plasma or a part or fraction thereof. In other embodiments, the sample is a blood or serum and the markers are proteins measured therefrom. In yet another embodiment, the sample is a tissue section and the markers are mRNA expressed therein. Many other combinations of sample forms from the human subjects and the form of the markers are contemplated.

Many markers are known for diseases, including cancers and a known panel can be selected, or as was done by the present Applicants, a panel can be selected based on measurement of individual markers in longitudinal clinical samples wherein a panel is generated based on empirical data for a desired disease such as cancer.

Examples of biomarkers that can be employed include molecules detectable, for example, in a body fluid sample, such as, antibodies, antigens, small molecules, proteins, hormones, enzymes, genes and so on. However, the use of tumor antigens has many advantages due to their widespread use over many years and the fact that validated and standardized detection kits are available for many of them for use with the aforementioned automated immunoassay platforms.

In embodiments, a panel of biomarkers are selected from AFP, CEA, CA125, CA19-9, CA 15-3, CYFRA21-1, PSA and SCC. In certain embodiments, the panel of biomarkers is selected from anti-p53, anti-NY-ESO-1, anti-ras, anti-Neu, anti-MAPKAPK3, cytokeratin 8, cytokeratin 19, cytokeratin 18, CEA, CA125, CA15-3, CA19-9, Cyfra 21-1, serum amyloid A, proGRP and 1-anti-trypsin (US20120071334; US20080160546; US20080133141; US 20070178504 (each herein incorporated by reference)). Additional tumor markers include human epididymal protein 4; calcitonin, PAP, BR 27.29, Her-2; and HE-4.

Autoantibodies that are proposed to be circulating markers for lung cancer include p53, NY-ESO-1, CAGE, GBU4-5, Annexin 1, SOX2 and IMPDH, phosphoglycerate mutase, ubiquillin, Annexin I, Annexin II, and heat shock protein 70-9B (HSP70-9B).

In certain embodiments, a panel of markers comprises markers associated with a cancer selected from bile duct cancer, bone cancer, pancreatic cancer, cervical cancer, colon cancer, colorectal cancer, gallbladder cancer, liver or hepatocellular cancer, ovarian cancer, testicular cancer, lobular carcinoma, prostate cancer, and skin cancer or melanoma. In other embodiments, a panel of markers comprises markers associated with breast cancer. In certain embodiment, a panel of biomarkers comprises markers associated with “pan-cancer”.

In certain regions of the world, most notably in the Far East, many hospitals and “Health Check Centers” offer panels of tumor markers to patients as part of their annual physicals or check-ups. These panels are offered to patients without noticeable signs or symptoms of, or predisposition to, any particular cancer and are not specific to any one tumor type (i.e. “pan-cancer”). Exemplary of such testing approaches is the one reported by Y.-H. Wen et al., Clinica Chimica Acta 450 (2015) 273-276, “Cancer Screening Through a Multi-Analyte Serum Biomarker Panel During Health Check-Up Examinations: Results from a 12-year Experience.” The authors report on the results from over 40,000 patients tested at their hospital in Taiwan between 2001 and 2012. The patients were tested with the following biomarkers: AFP, CA 15-3, CA125, PSA, SCC, CEA, CA 19-9, and CYFRA, 21-1 using kits available from Roche Diagnostics, Abbott Diagnostics, and Siemens Healthcare Diagnostics. The sensitivity of the panel for identifying the four most commonly diagnosed malignancies in that region (i.e. liver cancer, lung cancer, prostate cancer, and colorectal cancer) was 90.9%, 75.0%, 100% and 76%, respectively. Subjects with at least one of the markers showing values above the cut-off point were considered positive for the assay. No algorithm was reported. Moreover, neither clinical parameters nor biomarker velocity were factored in with this test.

It is believed that the methods and machine learning systems according to the present invention can improve and enhance the pan-cancer biomarker panel reported by the Taiwanese group and readily permit its use in other parts of the world. For example, an algorithm that combines biomarker values with clinical parameters could be employed that automatically improves using the machine learning software.

A panel can comprise any number of markers as a design choice, seeking, for example, to maximize specificity or sensitivity of the classifier model. Hence, the present methods may ask for presence of at least one of two or more biomarkers, three or more biomarkers, four or more biomarkers, five or more biomarkers, six or more biomarkers, seven or more biomarkers, eight biomarkers or more as a design choice.

Thus, in one embodiment, the panel of biomarkers may comprise at least two, at least three, at least four, at least five, at least six, at least seven, at least eight, at least nine or at least ten or more different markers. In one embodiment, the panel of biomarkers comprises about two to ten different markers. In another embodiment, the panel of biomarkers comprises about four to eight different markers. In yet another embodiment, the panel of markers comprises about six or about seven different markers.

Generally, a sample is committed to the assay and the results can be a range of numbers reflecting the presence and level (e.g., concentration, amount, activity, etc.) of presence of each of the biomarkers of the panel in the sample.

The choice of the markers may be based on the understanding that each marker, when measured and normalized, contributed equally as an input variable for the classifier model. Thus, in certain embodiments, each marker in the panel is measured and normalized wherein none of the markers are given any specific weight. In this instance each marker has a weight of 1.

In other embodiments, the choice of the markers may be based on the understanding that each marker, when measured and normalized, contributed unequally as an input variable for the classifier model. In this instance, a particular marker in the panel can either be weighted as a fraction of 1 (for example if the relative contribution is low), a multiple of 1 (for example if the relative contribution is high) or as 1 (for example when the relative contribution is neutral compared to the other markers in the panel).

In still other embodiments, a machine learning system may analyze values from biomarker panels without normalization of the values. Thus, the raw value obtained from the instrumentation to make the measurement may be analyzed directly.

The use in a clinical setting of the embodiments presented herein are now described in the context of “pan-cancer” and specific cancer screening.

Primary care healthcare practitioners, who may include physicians specializing in internal medicine or family practice as well as physician assistants and nurse practitioners, are among the users of the techniques disclosed herein. These primary care providers typically see a large volume of patients each day. In one instance these patients are at risk for lung cancer due to smoking history, age, and other lifestyle factors. In 2012 about 18% of the U.S. population was current smokers and many more were former smokers with a lung cancer risk profile above that of a population that has never smoked.

A blood sample from patient, such as a patient 50 years of age or older, is sent to a laboratory qualified to test the sample using a panel of biomarkers, such as those used to train the present classifier models generated by a machine learning system. Non-limiting lists of such biomarkers are herein included throughout the specification including the examples. In lieu of blood, other suitable bodily fluids such a sputum or saliva might also be utilized.

The measured values of the biomarkers are then used as input values, along with age, to be used with the first classifier model in a computer implemented system. An output value is obtained and compared to a threshold value wherein the threshold is empirically determined and set to separate patients in a low risk category from those in an increased risk for having or developing cancer. The threshold value is empirically determined using longitudinal clinical data. If the risk calculation is to be made at the point of care, rather than at the laboratory, a software application compatible with mobile devices (e.g. a tablet or smart phone) may be employed.

For those patients classified into an increased risk category, the input variables of measured biomarkers and age may be used with the second classifier model in a computer implemented system. An output value is obtained and compared to the longitudinal clinical data used to train the second classifier model and assigned a class membership, wherein the class memberships are organ system. In certain embodiments, the class membership is further defined by a specific cancer type, e.g. lung cancer.

Once the physician or healthcare practitioner has a risk score for the patient (i.e. risk that the patient has or will develop cancer relative to a population of others with comparable epidemiological factors) and the most likely organ malignancy or specific cancer, follow-up testing can be recommended for those at higher risk, such as radiography screening or tissue biopsy. It should be appreciated that the precise numerical cut off above which further testing is recommended may vary depending on many factors including, without limitation, (i) the desires of the patients and their overall health and family history, (ii) practice guidelines established by medical boards or recommended by scientific organizations, (iii) the physician's own practice preferences, and (iv) the nature of the biomarker test including its overall accuracy and strength of validation data.

It is believed that use of the embodiments presented herein will have the twin benefits of ensuring that the most at-risk patients undergo further diagnostic testing so as to detect early tumors and occult cancer that can be cured with surgery while reducing the expense and burden of false positives associated with stand-alone screening.

Embodiments of the present invention further provide for an apparatus for assessing a subject's risk level for the presence of cancer and correlating the risk level with an increase or decrease of the presence of cancer after testing relative to a population or a cohort population. The apparatus may comprise a processor configured to execute computer readable media instructions (e.g., a computer program or software application, e.g., a machine learning system, to receive the concentration values from the evaluation of biomarkers in a sample and, in combination with other risk factors (e.g., medical history of the patient, publicly available sources of information pertaining to a risk of developing cancer, etc.) may determine a risk score and compare it to a grouping of stratified cohort population comprising multiple risk categories.

The apparatus can take any of a variety of forms, for example, a handheld device, a tablet, or any other type of computer or electronic device. The apparatus may also comprise a processor configured to execute instructions (e.g., a computer software product, an application for a handheld device, a handheld device configured to perform the method, a world-wide-web (WWW) page or other cloud or network accessible location, or any computing device. In other embodiments, the apparatus may include a handheld device, a tablet, or any other type of computer or electronic device for accessing a machine learning system provided as a software as a service (SaaS) deployment. Accordingly, the correlation may be displayed as a graphical representation, which, in some embodiments, is stored in a database or memory, such as a random access memory, read-only memory, disk, virtual memory, etc. Other suitable representations, or exemplifications known in the art may also be used.

The apparatus may further comprise a storage means for storing the correlation, an input means, and a display means for displaying the status of the subject in terms of the particular medical condition. The storage means can be, for example, random access memory, read-only memory, a cache, a buffer, a disk, virtual memory, or a database. The input means can be, for example, a keypad, a keyboard, stored data, a touch screen, a voice-activated system, a downloadable program, downloadable data, a digital interface, a hand-held device, or an infrared signal device. The display means can be, for example, a computer monitor, a cathode ray tube (CRT), a digital screen, a light-emitting diode (LED), a liquid crystal display (LCD), an X-ray, a compressed digitized image, a video image, or a hand-held device. The apparatus can further comprise or communicate with a database, wherein the database stores the correlation of factors and is accessible to the user.

In another embodiment of the present invention, the apparatus is a computing device, for example, in the form of a computer or hand-held device that includes a processing unit, memory, and storage. The computing device can include or have access to a computing environment that comprises a variety of computer-readable media, such as volatile memory and non-volatile memory, removable storage and/or non-removable storage. Computer storage includes, for example, RAM, ROM, EPROM & EEPROM, flash memory or other memory technologies, CD ROM, Digital Versatile Disks (DVD) or other optical disk storage, magnetic cassettes, magnetic tape, magnetic disk storage or other magnetic storage devices, or other medium known in the art to be capable of storing computer-readable instructions. The computing device can also include or have access to a computing environment that comprises input, output, and/or a communication connection. The input can be one or several devices, such as a keyboard, mouse, touch screen, or stylus. The output can also be one or several devices, such as a video display, a printer, an audio output device, a touch stimulation output device, or a screen reading output device. If desired, the computing device can be configured to operate in a networked environment using a communication connection to connect to one or more remote computers. The communication connection can be, for example, a Local Area Network (LAN), a Wide Area Network (WAN) or other networks and can operate over the cloud, a wired network, wireless radio frequency network, and/or an infrared network.

Artificial intelligence systems include computer systems configured to perform tasks usually accomplished by humans, e.g., speech recognition, decision making, language translation, image processing and recognition, etc. In general, artificial intelligence systems have the capacity to learn, to maintain and access a large repository of information, to perform reasoning and analysis in order to make decisions, as well as the ability to self-correct.

Artificial intelligence systems may include knowledge representation systems and machine learning systems. Knowledge representation systems generally provide structure to capture and encode information used to support decision making. Machine learning systems are capable of analyzing data to identify new trends and patterns in the data. For example, machine learning systems may include neural networks, induction algorithms, genetic algorithms, etc. and may derive solutions by analyzing patterns in data.

In certain embodiments, the present classifier models comprise an algorithm such as a support vector machine, a decision tree, a random forest, a neural network, a deep learning neural network, a logistic regression or a pattern recognition algorithm. The present classifier models may be used to classify an individual patient into one of a plurality of categories, e.g., a category indicative of a likelihood of cancer or a category indicating that cancer is not likely. Inputs to the classifier model may include a panel of biomarkers associated with the presence of cancer as well as clinical parameters. See Example 3. In embodiments, clinical parameters include one or more of the following: (1) age; (2) gender; (3) smoking history in years; (4) number of packs per year; (5) symptoms; (6) family history of cancer; (7) concomitant illnesses; (8) number of nodules; (9) size of nodules; and (10) imaging data and so forth. In exemplary embodiments, the clinical parameter used as in put value is age wherein gender is used to train the classifier model providing a classifier model for male patients and a separate classifier model for female patients.

In certain embodiments, the clinical parameters include smoking history in years, number of packs per year, and age. In still other embodiments, the panel of biomarkers comprises any two, any three, any four, any five, any six, any seven, any eight, any nine, or any ten biomarkers. In embodiments, the panel of biomarkers comprises two or more biomarkers selected from the group consisting of: AFP, CA125, CA 15-3, CA 19-19, CEA, CYFRA 21-1, HE-4, NSE, Pro-GRP, PSA, SCC, anti-Cyclin E2, anti-MAPKAPK3, anti-NY-ESO-1, and anti-p53. In other embodiments, the panel of biomarkers comprises CA 19-9, CEA, CYFRA 21-1, NSE, Pro-GRP, and SCC. In still other embodiments, the panel of biomarkers comprises AFP, CA125, CA 15-3, CA-19-9, CEA, HE-4, and PSA. In yet other embodiments, the panel of biomarkers comprises AFP, CA125, CA 15-3, CA-19-9, Calcitonin, CEA, PAP, and PSA. In other embodiments, the panel of biomarkers comprises AFP, BR 27.29, CA12511, CA 15-3, CA-19-9, Calcitonin, CEA, Her-2, and PSA.

A variety of machine learning models are available, including support vector machines, decision trees, random forests, neural networks or deep learning neural networks. Generally, support vector machines (SVMs) are supervised learning models that analyze data for classification and regression analysis. SVMs may plot a collection of data points in n-dimensional space (e.g., where n is the number of biomarkers and clinical parameters), and classification is performed by finding a hyperplane that can separate the collection of data points into classes. In some embodiments, hyperplanes are linear, while in other embodiments, hyperplanes are non-linear. SVMs are effective in high dimensional spaces, are effective in cases in which the number of dimensions is higher than the number of data points, and generally work well on data sets with clear margins of separation.

Decision trees are a type of supervised learning algorithm also used in classification problems. Decision trees may be used to identify the most significant variable that provides the best homogenous sets of data. Decision trees split groups of data points into one or more subsets, and then may split each subset into one or more additional categories, and so forth until forming terminal nodes (e.g., nodes that do not split). Various algorithms may be used to decide where a split occurs, including a Gini Index (a type of binary split), Chi-Square, Information Gain, or Reduction in Variance. Decision trees have the capability to rapidly identify the most significant variables among a large number of variables, as well as identify relationships between two or more variables. Additionally, decision trees can handle both numerical and non-numerical data. This technique is generally considered to be a non-parametric approach, e.g., the data does not have to fit a normal distribution.

Random forest (or random decision forest) is a suitable approach for both classification and regression. In some embodiments, the random forest method constructs a collection of decision trees with controlled variance. Generally, for M input variables, a number of variables (nvar) less than M is used to split groups of data points. The best split is selected and the process is repeated until reaching a terminal node. Random forest is particularly suited to process a large number of input variables (e.g., thousands) to identify the most significant variables. Random forest is also effective for estimating missing data.

Neural nets (also referred to as artificial neural nets (ANNs)) are described throughout this application. A neural net, which is a non-deterministic machine learning technique, utilizes one or more layers of hidden nodes to compute outputs. Inputs are selected and weights are assigned to each input. Training data is used to train the neural networks, and the inputs and weights are adjusted until reaching specified metrics, e.g., a suitable specificity and sensitivity.

ANNs may be used to classify data in cases in which correlation between dependent and independent variables is not linear or in which classification cannot be easily performed using an equation. More than 25 different types of ANNs exist, with each ANN yielding different results based on different training algorithms, activation/transfer functions, number of hidden layers, etc. In some embodiments, more than 15 types of transfer functions are available for use with the neural network. Prediction of the likelihood of having cancer is based upon one or more of the type of ANN, the activation/transfer function, the number of hidden layers, the number of neurons/nodes, and other customizable parameters.

Deep learning neural networks, another machine learning technique, are similar to regular neural nets, but are more complex (e.g., typically have multiple hidden layers) and are capable of automatically performing operations (e.g., feature extraction) in an automated manner, generally requiring less interaction with a user than a traditional neural net.

In some embodiments, inputs may be selected in order to improve the performance of the classifier model. For example, rather than picking the set of inputs that achieves the highest possible sensitivity with a clinically relevant specificity such as 80% or greater, the inputs are selected to reach a sensitivity threshold (e.g., 80% or greater), and once reaching this threshold, the inputs are selected to optimize performance of the classifier model, thereby improving the performance of the classifier model.

Accordingly, systems, methods and computer readable media are presented herein regarding using a machine learning system, e.g. to generate a classifier model, to identify a patient's risk of having cancer. A set of data comprising a plurality of patient records, each patient record including a plurality of parameters and corresponding values for a patient, and wherein the set of data also includes a diagnostic indicator indicating whether or not the patient has been diagnosed with cancer is stored in a memory, accessible by the classifier model or machine learning system. The plurality of parameters includes various biomarkers, clinical factors and other factors which may be selected as inputs into the classifier model. The diagnostic indicator is an affirmative indicator that the patient has cancer, e.g., a lung X-ray and/or biopsy confirming a diagnosis of cancer. A subset of the plurality of parameters is selected for inputs into the machine learning system, wherein the subset includes a panel of at least two different biomarkers and at least one clinical parameter, such as age.

In order to train the classifier model generated by the machine learning system, the set of data (e.g. longitudinal) is randomly partitioned into training data and validation data. The classifier model is generated using the machine learning system based on the training data, the subset of inputs and other parameters associated with the machine learning system as described herein. It is determined whether the classifier meets certain performance criteria, such as a predetermined Receiver Operator Characteristic (ROC) statistic, specifying a sensitivity and a specificity, for correct classification of patients. In embodiments, the specificity is at least 80% and the sensitivity is at least 75%. See Example 1A and 2.

When the classifier model does not meet the predetermined ROC statistic, the classifier may be iteratively regenerated based on the training data and a different subset of inputs until the classifier meets the pre-determined ROC statistic. When the machine learning system meets the predetermined ROC statistic, a static configuration of the classifier may be generated. This static configuration may be deployed to a physician's office for use in identifying patients at risk of having lung cancer or stored on a remote server that can be accesses by the physician's office.

Once the classifier model has been trained on the training data, the classifier model may be validated using the validation data. The validation data also includes a plurality of parameters and corresponding values for a patient, and includes a diagnostic indicator indicating whether or not the patient has been diagnosed with cancer. The validation data may be classified using the classifier model, and it may be determined whether the classifier meets the predetermined performance criteria such as a ROC statistic based on this data. When the classifier model does not meet the predetermined ROC statistic, the classifier may be iteratively regenerated based on the training data and a different subset of the plurality of parameters, until the regenerated classifier meets the predetermined ROC statistic. The validation process may then be repeated.

A user, with access to a computing device with the static classifier model, may enter input values corresponding to a patient into the computing device. The patient may then be classified, using the static classifier, into a risk category indicative of a likelihood of having cancer or into another risk category indicative of a likelihood of not having cancer. The system may then send a notification to the user (e.g., a physician) recommending additional diagnostic testing (e.g., a CT scan, a chest x-ray or biopsy) when the patient is classified into the category indicative of a likelihood of having cancer.

In some embodiments, the classifier model generated by the machine learning system may be continuously trained over time. Test results obtained from the diagnostic testing, which confirm or deny the presence of cancer, may be incorporated into the training data set for further training of the machine learning system, and to generate an improved classifier by the machine learning system.

Thus, in some embodiments, the values of a panel of biomarkers in a sample from a patient are measured. A classifier model is generated by a machine learning system to classify the patient into a risk category for having or developing cancer, wherein the classifier model has a performance of a ROC curve with a sensitivity of at least 80% and a specificity of at least 80% for correct classification of the patient as having or developing cancer, and wherein the classifier is generated using the panel of biomarkers comprising at least two different biomarkers, and at least one clinical parameter, such as age. When a patient is classified into an increased risk category for having or developing cancer, a notification to a user for diagnostic testing is provided. In embodiments, the risk category for having or developing cancer may be further categorized into qualitative groups (e.g. high, low, medium, etc.) for the likelihood of having cancer, or into quantitative groups (e.g. a percentage, multiplier, risk score, composite score) of the likelihood of having cancer.

In certain embodiments, for patients classified into an increased risk category for having or developing cancer, a second classifier model is generated by a machine learning system to assign patients to an organ system and/or specific cancer class membership, wherein the classifier model has a performance of a ROC curve with a sensitivity of at least 70% and a specificity of at least 80% for correct classification of the patient as having or developing cancer, and wherein the classifier is generated using the panel of biomarkers comprising at least two different biomarkers, and at least one clinical parameter, such as age. Following classification into a class membership, a notification to a user for diagnostic testing is provided.

In other embodiments, a computer implemented method for predicting a risk or having or developing cancer in a subject, using a computer system having one or more processors coupled to a memory storing one or more computer readable instructions for execution by the one or more processors, the one or more computer readable instructions comprising instructions for: storing a set of data comprising a plurality of patient records, each patient record including a plurality of parameters for a patient, and wherein the set of data also includes a diagnostic indicator indicating whether or not the patient has been diagnosed with cancer; selecting a plurality of parameters for inputs into a machine learning system, wherein the parameters include a panel of at least two different biomarker values and at least one type of clinical data; and generating a classifier using the machine learning system, wherein the classifier comprises a sensitivity of at least 70% and a specificity of at least 80%, and wherein the classifier is based on a subset of the inputs.

In some embodiments, although the machine learning system can evolve over time to make more accurate predictions, the machine learning system may have the capability to deploy improved predictions on a scheduled basis. In other words, the techniques used by the machine learning system to determine risk may remain static for a period of time, allowing consistency with regard to determination of a risk score. At a specified time, the machine learning system may deploy updated techniques that incorporate analysis of new data to produce an improved risk score. Thus, the machine learning systems described herein may operate: (1) in a static manner; (2) in a semi-static manner, in which the classifier is updated according to a prescribed schedule (e.g., at a specific time); or (3) in a continuous manner, being updated as new data is available.

### EXAMPLES

The Examples below are given so as to illustrate the practice of this invention. They are not intended to limit or define the entire scope of this invention.

### Example 1A: Development of a Multi-Marker Model for Classifying Asymptomatic Patients as to Developing Cancer: “Pan-Cancer” Test

Provided herein is a multi-marker classification model and method for identifying asymptomatic patients with an increased risk for developing cancer. That risk can be categorized as “low”, “medium/moderate” or “high risk” for developing cancer, wherein the ranges for those categories may be based on, for example, probability of developing cancer within 6 months to a year, wherein the probability is measured against baseline level of cancer in the heterogenous population. It is understood in the art, that the rate of cancer is about 1% in the general population. The prevalence of cancer in the cohort used to develop the present pan-cancer_test was about 1.5%. See the below examples for more detail on the use of the test and probability values. The development of the classifier model, and the selection of markers (both blood and clinical parameters) may be based on a combination of accuracy, area under the curve (AUC), sensitivity, specificity values, and/or Youden index (Sensitivity+Specificity−1) that provide a measure of the performance of the classifier model.

The development and continued learning by the classifier model of the pan-cancer Test was performed using longitudinal data and/or retrospective data over a 12-year period wherein biomarkers were measured (along with gender and age), statistical analysis performed, and that data correlated to those individuals that developed cancer. From that, a model comprising an algorithm was generated and trained to identify those individuals with an increased risk at developing cancer over the following 6 months to a year. The same principal is applied to continually increase the accuracy of the model wherein individuals and their biomarker measurements are added to the cohort and further train the model.

The present “pan-cancer” model was developed using data from 12,622 asymptomatic males and 15,316 asymptomatic females who had sera biomarkers measured based on a tumor marker panel over a 12-year period in Taiwan. The male cohort had a panel of six markers measured (AFP, CEA, CA19-9, CA15-3, CA125, PSA, SCC, and CYFRA21-1) and the female cohort had a panel of seven markers measured (AFP, CEA, CA19-9, CA125, CA15-3, SCC, and CYFRA21-1). All tumor markers were measured using commercially available in vitro diagnostic (IVD) kits and instrumentation manufactured by either Roche or Abbott Diagnostics. All assays of tumor markers met the requirements of the College of American Pathologists (CAP) Laboratory Accreditation Program. Outcome data were obtained from a cancer registry to determine whether each patient had received a new diagnosis of malignancy within 1 year of the tumor markers test.

All 27,938 individuals were randomly allocated to the training (⅔) or testing (⅓) set. All randomizations were performed using Matlab (Math-Works, Natick, MA, USA).

Because of the unbalanced nature of the data sets (far greater number of non-cancers vs. true cancers) used in this study, data reprocessing was performed to improve the selection of negative samples using a stratified sampling technique. A cancer to noncancer ratio of 1:1 was adopted to randomize 124 males and 104 females from the 8291 and 10107 noncancer cases, respectively, to the final training set. Consequently, the training sets that comprised 124 cases of newly diagnosed cancer and 124 noncancer cases for males and 104 cancers and 104 noncancer cases for females were used to train the machine learning models.

Statistical Analysis. The biomarker panel AFP, CEA, CA19-9, CYFRA21-1, SCC and PSA were measured for all 12,622 male individuals and the biomarker panel AFP, CEA, CA19-9, CA125, CA15-3, SCC, and CYFRA21-1 were measured for all 15,316 female individuals. A variable selection process was applied to select robust variables from those serum tumor markers to design cancer detection models. The accuracy, sensitivity, specificity, AUC (area under the curve), and Youden index were compared to select the best machine learning models.

The Youden index was used as a performance indicator for selecting the variables used in the classifier models in this study. The Youden index, which is among the most widely used performance indicators in biomedical studies, is calculated using the following formula:

Youden index=Sensitivity+Specificity−1.

Statistical Algorithms and Models for Cancer Screening. In this study, multiple cancer screening models using the above measured serum tumor markers were designed using machine learning methods, including: SVM, kNN, MLR, Sequential Minimal Optimization (SMO), J48 decision tree, Neighborhood-Based Clustering Algorithm (NBC), Library for Support Vector Machines LibSVM, Ensemble Vote Classifier (LibSVM, LR, NBC), and Multilayer Perceptron (MLP).

Results. To design cancer detection models using machine learning methods and the panel of six biomarkers measured in the male cohort, 63 combinations of tumor markers were evaluated using the Youden index to select an appropriate combination of variables for constructing effective cancer classification models with the highest AUC and/or Youden Index. ROC curves and AUC values were used to assess the performance of the various machine learning methods for cancer prediction. Those results are provided below in Table 1.

TABLE 1
Comparison of Various Methods for Cancer Screening
(Male) using a model that includes all 6 biomarkers
(AFP, CEA, CA19-9, CYFRA21-1, PSA and SCC) and age

| | | | | | Youden |
|---|---|---|---|---|---|
| Classifier | Accuracy | AUC | Sensitivity | Specificity | Idx |
| LibSVM (RBF) | 64.94% | 0.695 | 0.742 | 0.648 | 0.390 |
| SMO (PolyKernel) | 80.87% | 0.816 | 0.823 | 0.808 | 0.631 |
| KNN (k = 15) | 75.90% | 0.839 | 0.790 | 0.759 | 0.549 |
| J48 decision tree | 85.64% | 0.760 | 0.484 | 0.862 | 0.346 |
| NBC | 96.79% | 0.826 | 0.210 | 0.979 | 0.189 |
| Logistic Regression | 76.87% | 0.870 | 0.823 | 0.768 | 0.591 |
| (Simple) | | | | | |
| Ridge Logistic | 80.44% | 0.874 | 0.823 | 0.804 | 0.627 |
| Regression | | | | | |
| Vote (LibSVM, | 82.91% | 0.839 | 0.677 | 0.831 | 0.508 |
| LR, NBC) | | | | | |
| MLP | 68.70% | 0.868 | 0.871 | 0.684 | 0.555 |

The AUC values for all various machine learning methods that integrated multiple biomarkers outperformed the individual biomarker AUC values, as previously published (Wen Y H, Chang P Y, Hsu C M, Wang H Y, Chiu C T, Lu J J. (2015) Cancer screening through a multi-analyte serum biomarker panel during health check-up examinations: Results from a 12-year experience. Clinica chimica acta, International Journal of Clinical Chemistry 450:273-6; Wang H Y, Hsieh C H, Wen C N, Wen Y H, Chen C H, Lu J J (2016) Cancer Screening in an Asymptomatic Population by Using Multiple Tumour Markers. PLOS ONE 11(6)). That was further validated comparing the single threshold method for individual biomarkers to the present classifier model with the same data set. See Example 4 and 5.

For male individuals, the SVM (SMO, PolyKernel, no normalization) model that combined all 6 biomarkers (AFP, CEA, CA19-9, CYFRA21-1, PSA and SCC) and age attained the highest Youden Index (0.631) (Table 1). However, the highest AUC was achieved for Ridge Logistic Regression model that incorporated the same variables−6 biomarkers and age (Table 1).

Leaving out any one marker had minimal negative effect on the performance of the SMO model, either Youden Index or AUC (Table 2). Similar trend was observed for the Ridge Logistic Regression model with exception of SCC biomarker omission that had no effect on the LR model performance (Table 3).

TABLE 2
Leave-one-out analysis using SMO (PolyKernel) (male model).

| | | | | | Youden |
|---|---|---|---|---|---|
| SMO (PolyKernel) | Accuracy | AUC | Sensitivity | Specificity | Idx |
| 6-Biomarker + age | 80.87% | 0.816 | 0.823 | 0.808 | 0.631 |
| AFP | 79.46% | 0.808 | 0.823 | 0.794 | 0.617 |
| CA19-9 | 80.20% | 0.796 | 0.790 | 0.802 | 0.592 |
| CEA | 75.99% | 0.775 | 0.790 | 0.759 | 0.549 |
| CYFRA21-1 | 80.08% | 0.812 | 0.823 | 0.800 | 0.623 |
| PSA | 78.56% | 0.796 | 0.806 | 0.786 | 0.591 |
| SCC | 81.70% | 0.812 | 0.806 | 0.817 | 0.623 |

TABLE 3
Leave-one-out analysis using Ridge
Logistic Regression (male model)

| Ridge Logistic | | | | | Youden |
|---|---|---|---|---|---|
| Regression | Accuracy | AUC | Sensitivity | Specificity | Idx |
| 6-Biomarker + age | 80.44% | 0.874 | 0.823 | 0.804 | 0.627 |
| AFP | 79.27% | 0.877 | 0.823 | 0.792 | 0.615 |
| CA19-9 | 79.32% | 0.871 | 0.806 | 0.793 | 0.599 |
| CEA | 79.08% | 0.872 | 0.806 | 0.791 | 0.597 |
| CYFRA21-1 | 79.70% | 0.867 | 0.823 | 0.797 | 0.620 |
| PSA | 77.78% | 0.866 | 0.823 | 0.777 | 0.600 |
| SCC | 80.56% | 0.875 | 0.823 | 0.805 | 0.628 |

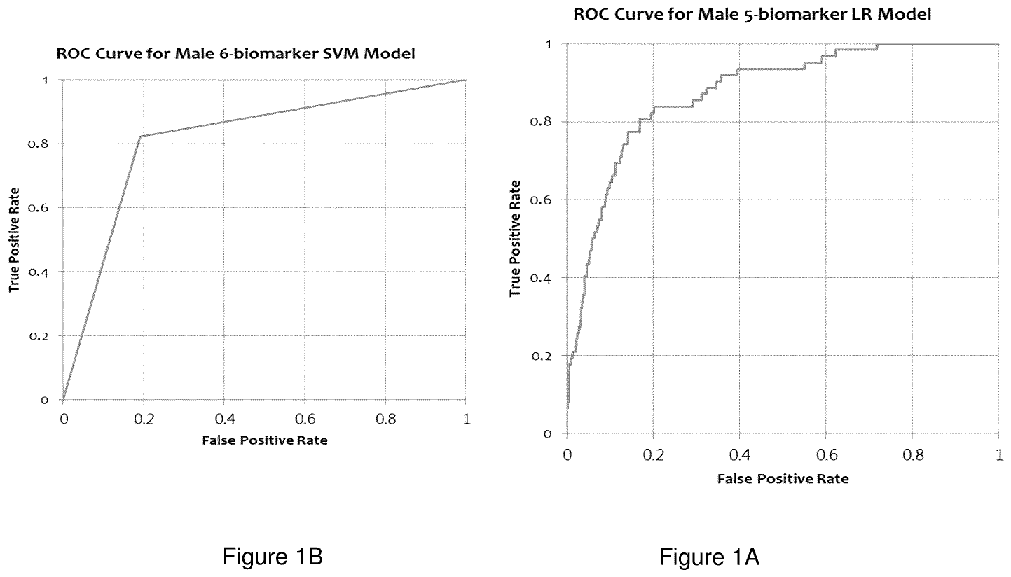

Based on the above results, the Logistic Regression model that included 5 tumor markers (without SCC) and age slightly outperformed SMO model (6 biomarkers and age) resulting in slightly higher AUC (0.875) and similar Youden Index (0.628). See FIG. 1 and Table 4.

TABLE 4
Performance of best cancer screening algorithms and models for males

| | | | | | | Youden |
|---|---|---|---|---|---|---|
| Model | Algorithm | Biomarkers | AUC | SE | SP | Index |
| 6-BM + age | SVM (SMO) | AFP, CEA, CA19-9, | 0.816 | 0.823 | 0.808 | 0.631 |
| | | CYFRA21-1, PSA and SCC | | | | |
| 5-BM + age | Ridge LR | AFP, CEA, CA19-9, | 0.875 | 0.823 | 0.805 | 0.628 |
| | | CYFRA21-1, PSA | | | | |
| Any BM high | None | AFP, CEA, CA19-9, | n/a | 0.515 | 0.851 | 0.366 |
| | | CYFRA21-1, PSA and SCC | | | | |

The same analysis as above was performed for the female cohort. However, the sensitivity and specificity of the machine learning SVM model were not as high as those for the male model. The performance of the best ML model for females (Vote (Lib SVM, LR, NBC)) was also greatly improved over the single threshold method (Youden Index 0.244 vs 0.028, respectively).

The ML models are amenable to periodic review and redefinition. Using a larger data set by combining the US and Asian cohorts, the accuracy of the pan-cancer model may be further improved for females by leveraging additional data and expanding the number of clinical factor predictors. It is also possible, without wishing to be bound by a theory, that a model for females may optionally account for fluctuations in hormones, such as during pregnancy or menstrual cycles, to further improve performance.

For individuals, female or male, the developed pan-cancer model can be applied to the panel of measured biomarkers, along with age and gender, to determine the likelihood that an individual is at risk for developing cancer. In certain embodiments, the time frame for developing cancer is a few months, such as within 3 months, and up to about 2 years. In certain embodiments, the “likelihood” an individual is at risk for developing cancer is a probability above background that the individual tested will develop cancer within a few months to about 2 years. For example, an individual may be classified as “moderate risk” wherein their probability of developing cancer is five times (5×) more than baseline, wherein baseline is about 1% in the general population. In other words, the likelihood a tested individual that is classified as “moderate risk” has a 5% risk of developing cancer as compared to a “low risk” individual that has a 1% risk of developing cancer over that same time period.

Accordingly, individuals identified as “moderate risk” or “high risk” may then be selected for further analysis for predicting organ system-based malignancy for a patient with an increased risk of having cancer. In certain embodiments, an individual with a probability above 0.5 (50%) using the selected model of Table 5, were classified as “moderate risk” or “high risk”. Individuals with a probability value below 0.5 (50%) were classified as “low risk”. The performance of the selected models had a sensitivity value of 0.82 and a specificity value of 0.81.

In certain embodiments, a method is provided for predicting an increased risk of having cancer for an asymptomatic patient, comprising measuring values of a panel of biomarkers in a sample from a patient; obtaining clinical parameters from the patient including age and gender; utilizing a classifier generated by a machine learning system to classify the patient into a low risk, moderate risk or high risk category of having or developing cancer, wherein the classifier provides a probability value and those individuals with a probability of 0.5 or greater are classified as moderate risk or high risk, and wherein the classifier is generated using a panel of at least six biomarkers, age, gender and a diagnostic indicator from a plurality of patient records and wherein the classifier has a performance based on a Receiver Operator Characteristic (ROC) curve of a sensitivity value of at least 0.8 and a specificity value of at least 0.8 for correct classification of the patient as having or developing cancer; and providing a notification to a user for diagnostic testing.

In embodiments, the present classifier model comprises the following importance factor for each variable, and for each gender.

TABLE A
Female Classifier Model

| Variable | Importance factor |
|---|---|
| Age | 9.1 |
| CYFRA21-1 | 7.6 |
| CEA | 6.4 |
| CA15-3 | 6.3 |
| CA125 | 5.8 |
| CA19-9 | 5.5 |
| AFP | 5.3 |

TABLE B
Male Classifier Model

| Variable | Importance factor |
|---|---|
| Age | 12.6 |
| PSA | 10.9 |
| CYFRA21-1 | 8.9 |
| CA19-9 | 8.1 |
| AFP | 7.8 |
| CEA | 7.5 |

### Example 1B: Improvement of a Multi-Marker Model for Classifying Asymptomatic Patients as to Developing Cancer: Inclusion of Clinical Factor “Age” in Model

Disclosed herein is an improved multi-marker model for classifying asymptomatic patients as to having or developing cancer. The above classifier model using only a panel of measured biomarkers was previously published wherein the performance of a Receiver Operating Characteristic (ROC) curve for the cohort of males was very low; sensitivity value of 0.515 and a specificity value of 0.851. The cohort of females had an even lower performance of a ROC curve with a sensitivity value of 0.345 and a specificity value of 0.880. See Tables 7 and 8 of Wang H. Y., Hsieh C. H., Wen C. N., Wen Y. H., Chen C. H. and Lu J. J., “Cancers Screening in an Asymptomatic Population by Using Multiple Tumour Markers” PLOS One, Jun. 29, 2016. In other words, the previous classifier model using only measured sera biomarkers was acceptable for excluding the risk of cancer for a patient with specificity values of at least 0.8. However, the previous classifier model was no better than 50% for predicting cancer, for males, and even worse than 50% for females. The performance of that model is un-usable in a clinical setting, wherein a classifier model needs to identify asymptomatic patients at risk for having or developing cancer as compared to other diagnostic means such as biopsy or radiography screens. As previously published, the classifier model using only measured sera biomarkers helped 1 in 125-200 males whereas 1 in 4-7 were harmed (false diagnosis); and, 1 in 200-333 females were helped whereas 1 in 3-8 females were harmed.

Applicants surprisingly found that including age in the classifier model as a variable significantly increased the performance of the classifier model. As disclosed in Example 1, age was used in the present classifier model along with the measured sera biomarkers AFP, CEA, CA19-9, CYFRA 21-1 and SCC along with PSA for men and CA 15-3 and CA125 for women. Table 1 shows a comparison of various models that includes all 6 biomarkers (AFP, CEA, CA19-9, CYFRA21-1, PSA and SCC) and age, wherein the classifier model performance was significantly increased with a sensitivity value of at least 0.8 and a specificity value of at least 0.8 (of a ROC curve).

### Example 2: Development of a Model for Predicting Organ System-Based Malignancy for Individuals in the “High Risk” and “Moderate Risk” Category Based on the Pan-Cancer Test

Provided herein are techniques for predicting organ system-based malignancy for a patient with an increased risk of having cancer as identified in Example 1. That information can then be used to refer patients to a specialist for more invasive diagnostic testing.

Using the entire cohort of cancer subjects (n=186) and the same six (or 5 for female individuals) biomarker measurements along with age and gender, we applied a model comprising a pattern recognition algorithm, and a k-Nearest Neighbors algorithm (kNN) employing a leave-one-out evaluation method to predict the top 1, 2, 3, 4, 5, 6, 7, 8, 9, or 10 cancers for each sample. The accuracies are reported in Table 5 and reflect the percentage of cases of each cancer type that were found in the top N (N=10 for Table 5) predicted cancers. Clearly, the accuracy of prediction varies based on both the cancer type and to some extent based on the number of cases of that type found in the dataset.

TABLE 5
Accuracy of Top N Cancer Type Model (males)

| Accuracy | Sample No. | Top 1 | Top 2 | Top 3 | Top 4 | Top 5 | Top 6 | Top 7 | Top 8 | Top 9 | Top 10 |
|---|---|---|---|---|---|---|---|---|---|---|---|
| All | 186 | 36.0% | 48.4% | 50.0% | 55.4% | 59.1% | 62.9% | 66.7% | 68.8% | 70.4% | 71.0% |
| Colon cancer | 20 | 15.0% | 25.0% | 30.0% | 45.0% | 50.0% | 60.0% | 75.0% | 75.0% | 80.0% | 80.0% |
| Kidney cancer | 12 | 25.0% | 50.0% | 50.0% | 50.0% | 58.3% | 66.7% | 66.7% | 75.0% | 75.0% | 75.0% |
| Liver cancer | 32 | 56.3% | 78.1% | 81.3% | 84.4% | 90.6% | 93.8% | 96.9% | 96.9% | 96.9% | 96.9% |
| Lung cancer | 10 | 30.0% | 40.0% | 40.0% | 40.0% | 50.0% | 50.0% | 50.0% | 50.0% | 60.0% | 60.0% |
| Pancreas cancer | 16 | 75.0% | 81.3% | 81.3% | 87.5% | 93.8% | 93.8% | 93.8% | 93.8% | 93.8% | 93.8% |
| Prostate cancer | 30 | 63.3% | 73.3% | 73.3% | 80.0% | 80.0% | 83.3% | 83.3% | 83.3% | 83.3% | 86.7% |

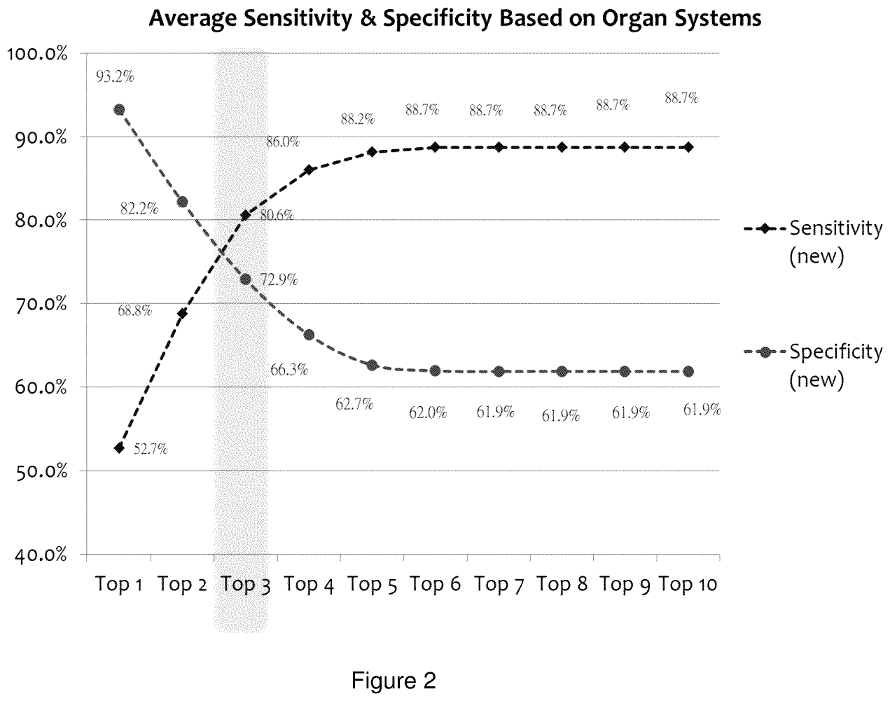

As such, it was decided to classify cancers more broadly based on organ system considering that would suggest the specialist to whom the patient should be referred. A similar analysis was performed, and the overall results are depicted in FIG. 2. A balanced sensitivity and specificity are achieved when the Top three most likely affected organ systems are reported. To a large extent the accuracies/sensitivities best reflect both the number of overall cases of a given cancer type in the dataset (i.e. Gastro-Intestinal (GI) and Genitourinary (GU) cancers vs. dermatological cancers) as well the nature of the biomarkers (e.g. PSA is specific for prostate and therefore GU.

TABLE 6

| | Representative Corresponding |
|---|---|
| Organ System | Cancer Type |
| Genitourinary (GU) | Bladder, Kidney, Prostate |
| Gastrointestinal (GI) | Liver (HCC), Colon (CRC), Stomach, |
| | Pancreatic, Esophagus, Bile Duct, |
| | Gastric |
| Pulmonary | Lung |
| Dermatological | Skin |
| Hematological | Leukemia, lymphoma, white blood |
| | cell cancers |
| Nervous System | Central Nervous System |
| Gynecological | Cervical, Ovary, Uterus |
| General | Breast, Liposarcoma |
| ENT | Head and Neck, Parotid, Thyroid |

When the selected model comprising pattern recognition algorithm, k-Nearest Neighbors algorithm (kNN), was used to determine the top three most likely organs to develop cancer in the “moderate risk” or “high risk” classified groups the performance of the test had a sensitivity value of 81% and the specificity value was 72%.

In certain embodiments, a method is provided for predicting organ system-based malignancy for a patient with an increased risk of having cancer, comprising: measuring values of a panel of biomarkers in a sample from a patient; obtaining clinical parameters from the patient including age and gender; utilizing a machine learning system to classify patient with an increased risk of having or developing cancer into an appropriate category, to identify at least one most likely organ system malignancy for that patient, wherein the classifier provides a class membership, and wherein the classifier is generated using a panel of at least six biomarkers, age, gender and a diagnostic indicator from a plurality of patient records and wherein the classifier has a performance based on a Receiver Operator Characteristic (ROC) curve of a sensitivity value of at least 0.8 and a specificity value of at least 0.7 for correct classification of the patient as having or developing cancer; and, providing a notification to a user for diagnostic testing.

### Example 3: Screening Patients for Likelihood of Developing Cancer and Predicting Mostly Likely Organ Involved in Cancer Using a Two-Step Model

Provided herein is a method for predicting organ system-based malignancy for a patient with an increased risk of having cancer, wherein a model trained from the cohort in Example 1 is applied to the measured panel of biomarkers and the clinical factors of age and gender to identify those patients with an increased risk of having or developing cancer; the pan-cancer test. Next, for those patients with a probability of an increased risk of having or developing cancer, 0.5 (50%), that are categorized as moderate or high risk, the model trained using the cohort of Example 2 is applied to the measured panel of biomarkers and the clinical factors of age and gender to provide a class membership (e.g. the organ system most likely (or top 2 or 3 organ systems)) to be involved in the cancer; the organ system-based malignancy test.

As disclosed in Example 2, the trained model predicts the top three organ systems. The output of the model may provide a class membership in one organ system (wherein the top three organ systems are all the same), in two organ systems (wherein two of the top three organ systems are the same) or in three organ systems (wherein the top three organ system predicted by the model are all different). See Table 6 for a list of organ systems (class membership) and representative caner types within each class.

In the present example, eight asymptomatic patients (5 male and 3 female) were first screened using the pan-cancer test according to Example 1, and then those categorized as moderate or high risk were further screened using the organ system-based malignancy test according to Example 2.

A panel of eight sera biomarkers were measured, with the exception that PSA was not measured in the female patients and CA 125 and/or CA 15-3 were not measured in male patients. See Table 7 below. For each patient, the following information was obtained:

General Information (age, gender, height, weight, race, ethnicity, current health status, fitness level)

Health History (Hypertension, Diabetes, Chronic Pancreatitis, Colorectal Polyps, Crohn's Disease, Ulcerative Colitis, COPD, Chronic Bronchitis, Emphysema, etc.)

Smoking History (pack years, smoking duration, age of smoking cessation)

Alcohol use (servings per week, duration)

For women only: childbirth and breastfeeding info, menstruation status, history of birth control pills, BRCA1, BRCA2, or other high-risk gene mutations (e.g., TP53, PALB2, CDH1, or ATM)

Cancer screening history (colonoscopy, sigmoidoscopy, mammogram, X-Ray or CT scan for Lung cancer, PAP/HPV test)

Cancer Family History (immediate family members diagnosed with any cancer)

See FIG. 3 for a table of the measured sera biomarker, age and gender used as variables for the input to the logistic regression algorithm used to provide a probability value. The probability values range from 0 to 1 and the probability ranges used to create the low, moderate and high-risk categories were different for the male and female patients. The current iteration of the application of the pan-cancer test model provides the following probability ranges for each category for male patients:
Low risk; 0 to 0.57
Moderate Risk; 0.58 to 0.79
High Risk; 0.8 to 1.

For a male patient with a probability value categorized as low risk, that means less than 1% of individuals with a probability value in that range will likely be found to have cancer. That risk level is no different than the general heterogeneous population; in other words, the low risk category represents no increased risk for a male patient as compared to baseline. For a male patient with a probability value categorized as moderate risk, that means approximately 5 out of 100 individuals with a probability value in that range were diagnosed with cancer within one year of having biomarkers measured. That risk level is approximately 5% of having or developing cancer within one year, or a five times (5×) increase as compared to the low risk category. For a male patient with a probability value categorized as high risk, that means approximately 10 out of 100 individuals with a probability value in that range were diagnosed with cancer within one year of having those biomarkers measured. That risk level is approximately 10% of having or developing cancer within one year, or a ten times (10×) increase as compared to the low risk category.

The current iteration of the application of the pan-cancer test model provides the following probability ranges for each category for female patients:
Low risk; 0 to 0.56×
Moderate Risk; 0.57 to 0.79
High Risk; 0.8 to 1.

For a female patient with a probability value categorized as low risk, that means less than 1% of individuals with a probability value in that range will likely be found to have cancer. That risk level is no different than the general heterogeneous population; in other words, the low risk category represents no increased risk for a female patient as compared to baseline. For a female patient with a probability value categorized as moderate risk, that means approximately 2 out of 100 individuals with a probability value in that range were diagnosed with cancer within one year of having biomarkers measured. That risk level is approximately 2% of having or developing cancer within one year, or a two times (2×) increase as compared to the low risk category. For a female patient with a probability value categorized as high risk, that means approximately 8 out of 100 individuals with a probability value in that range were diagnosed with cancer within one year of having those biomarkers measured. That risk level is approximately 8% of having or developing cancer within one year, or an eight times (8×) increase as compared to the low risk category.

One possible explanation for the discrepancy in increased risk between men and women with the application of the current model and biomarker measurements, is that up to 40% of diagnosed cancer in women is breast cancer, and as of today there are no good blood biomarkers that correlate with the presence of breast cancer.

Based on the risk category classification of the patients in FIG. 3, the trained pattern recognition model of Example 2 was applied to the high and moderate risk male patients and the high-risk female patient. Those same variables of FIG. 3 were used as input for the organ system-based malignancy test model. The output, a class membership of an organ system that represents a group of cancer types, may be used to suggest a specialist for follow-up care that may include radiography or invasive diagnostic tests.

Application of the organ system-based malignancy test model provided the following results:

TABLE 7

| | Organ System Class |
|---|---|
| Patient | Membership |
| Male #3 | Genitourinary (GU) |
| Male #4 | Gastrointestinal (GI) |
| Male #5 | Genitourinary (GU) and |
| | Gastrointestinal (GI) |
| Female #1 | Genitourinary (GU) |

In embodiments, a method is provided for predicting organ system-based malignancy for a patient with an increased risk of having cancer that utilizes a two-step machine learning process wherein a first machine learning model is applied using measured sera biomarkers and age as input variables, wherein gender is used to select the measured biomarkers and to train the classifier, to categorize patients as low risk (no increased risk) or moderate or high risk wherein the latter two categories represent an increased risk of having or developing cancer within one year as compared to baseline (low risk). For those patients categorized as moderate or high risk a second machine learning classifier is applied using the measured biomarkers, age and gender as input variables and providing a class membership for an organ system that represents a number of different cancer types.

In certain embodiments is provided a method for predicting organ system-based malignancy for a patient with an increased risk of having cancer, comprising: a) measuring values of a panel of biomarkers in a sample from a patient; b) obtaining clinical parameters from the patient including age and gender; c) utilizing a first classifier generated by a machine learning system to classify the patient into a low risk, moderate risk or high risk of having or developing cancer, wherein the classifier provides a probability value and those individuals with a probability of 0.5 or greater are classified as moderate risk or high risk, and wherein the classifier is generated using a panel of at least six biomarkers, age, gender and a diagnostic indicator from a plurality of patient records; utilizing a second classifier generated by a machine learning system, when a patient is classified into a medium or high risk category of developing cancer in step c), to identify at least one most likely organ system malignancy for that patient, wherein the classifier provides a class membership, and wherein the classifier is generated using a panel of at least six biomarkers, age, gender and a diagnostic indicator from a plurality of patient records; and, e) providing a notification to a user for diagnostic testing.

In some embodiments, the machine learning system comprises one or more machine learning processors. In other embodiments, the machine learning processors are deep learning processors. In other aspects, the one or more deep learning processors train one or more classification models using training data. In some aspects, the machine learning system generates one or more classifiers to predict a likelihood of having cancer or developing cancer, of class membership, or of both.

In some aspects, the machine learning model may comprise one or more classifiers, one or more inputs, and one or more weighting factors for weighting of the inputs, along with one or more classification models. The machine learning model may be continuously improved as new training data is available.

### Example 4: Male Classifier Model is Superior to a Single Threshold Method of Measuring Biomarkers for Prediction of Cancer

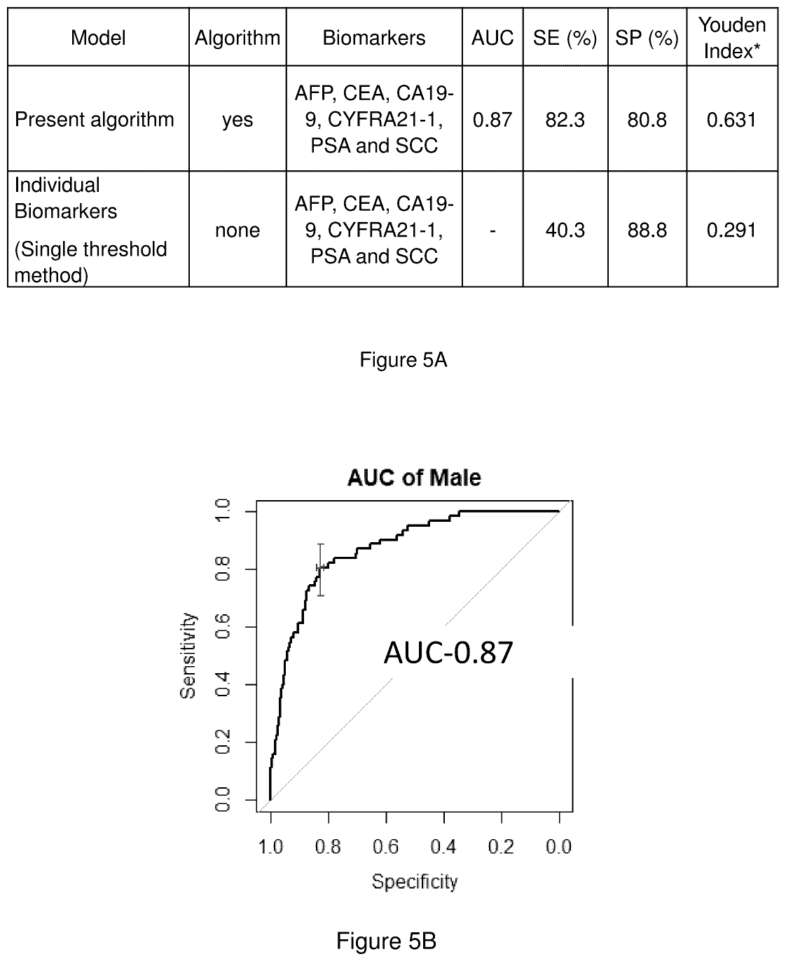

Provided herein is a demonstration that the present male classifier model, as developed in Example 1, is significantly better at predicting cancer development within one year than measurement of a panel of individual biomarkers from the same subjects. The present methods and classifier models aggregate biomarker measurements and clinical factors, such as age, to predict a patient's cancer risk, whereas previous methods may measure the same panel of markers but predict, or deem a patient an increased risk for developing cancer, if any one measured biomarker is “high”. In other words, any one biomarker above a threshold deemed to be clinically relevant would indicate a positive test for an increased risk of developing cancer. For example, Table 8 below provides a normal range for well-validated tumor markers, measurement of a given marker above the normal range would indicate an increased likelihood of developing cancer. The present male classifier model according to Example 1, and used in Example 3, provides a significant improvement to sensitivity and specificity for predicting cancer as compared to “any marker high” methods. See FIG. 5.

TABLE 8
Male Biomarkers with Well-Validated Performance:

| Biomarker | Normal Range | | Cancers |
|---|---|---|---|
| AFP | <8.3 | ng/ml | Liver cancer, testicular |
| | | | and ovarian cancers |
| CA 19-9 | <35 | U/ml | Pancreatic, colorectal, |
| | | | stomach, liver and bile |
| | | | duct cancer |
| CEA | <4.7 ng/ml | | Colorectal, pancreatic, |
| | (non-smokers) | | gastrointestinal cancers, |
| | <5.6 ng/ml | | lung cancer |
| | (smokers) | | |
| CYFRA 21-1 | <3.3 | ng/ml | Lung, H&N cancer, uterine |
| | | | cancer, esophagus cancer, |
| | | | bladder cancer, mesothelioma, |
| | | | some lymphomas and sarcomas |
| PSA | <4 | ng/ml | Prostate cancer |

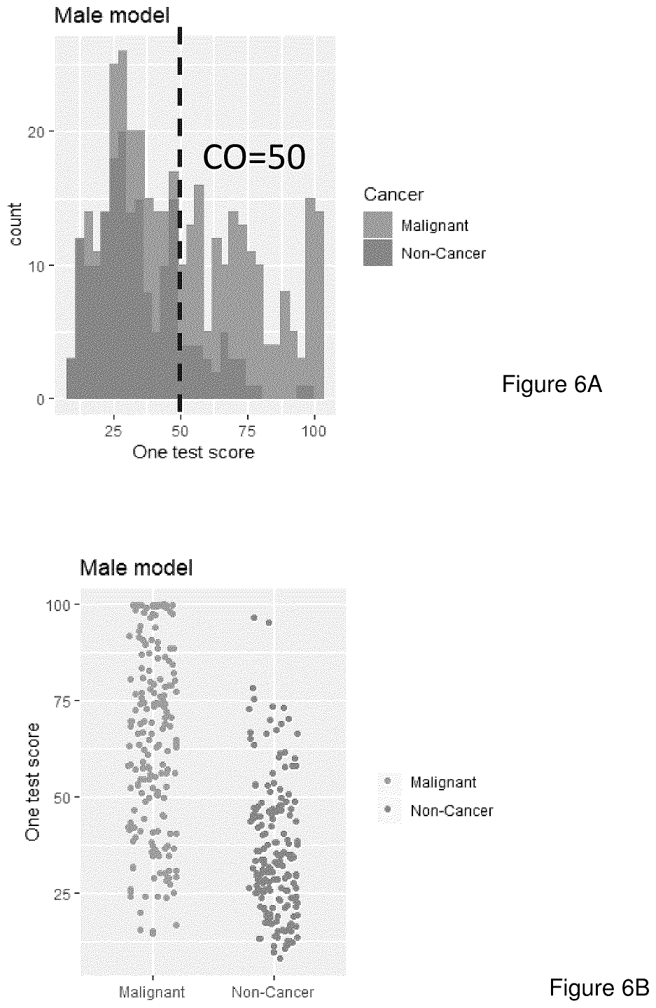

The present male classifier model provides a substantial improvement in diagnostic accuracy over conventional methods, e.g., any marker high methods; an improvement in sensitivity is demonstrated wherein 2× more cancers in males detected. Moreover, the present male classifier model was able to distinguish cancers from noncancers with 82% sensitivity and 81% specificity. See FIG. 6. In this figure, the cut off between low risk and moderate or high risk was 50, or 0.5. The risk score may be provided from 0 to 1, or 0 to 100.

### Example 5: Female Classifier Model is Superior to a Single Threshold Method of Measuring Biomarkers for Prediction of Cancer

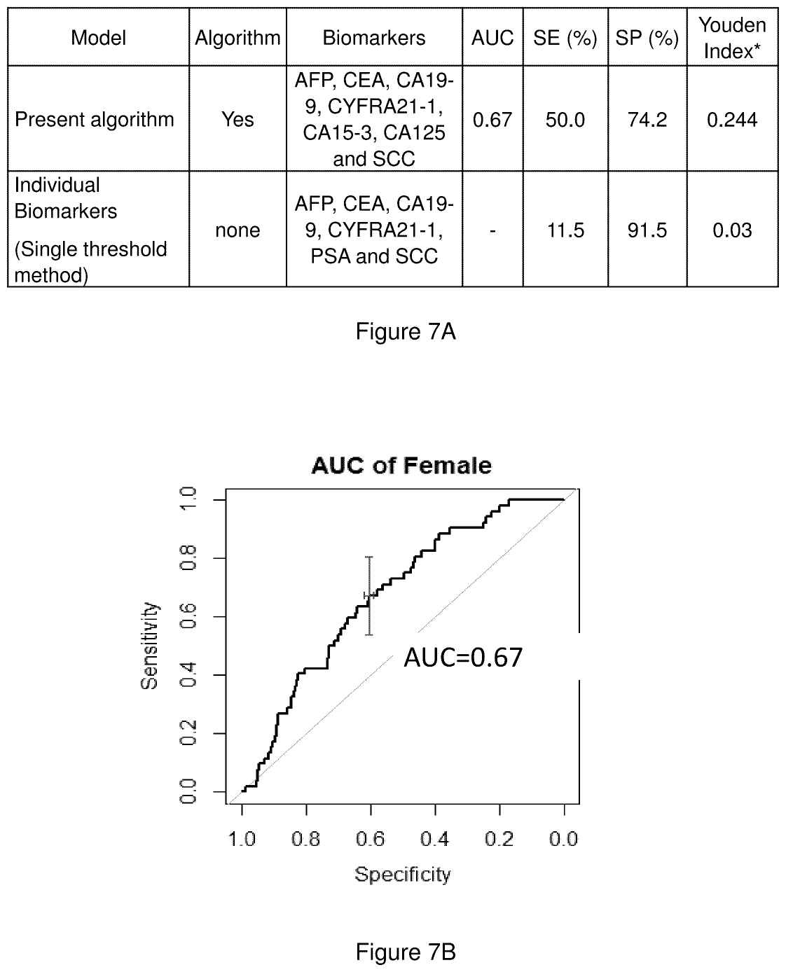

Provided herein is a demonstration that the present female classifier model, as developed in Example 1, is significantly better at predicting cancer development within one year than measurement of a panel of individual biomarkers from the same subjects. Notably, the present female classifier model improves individual biomarker “single threshold” method wherein the sensitivity represents a 4-fold increase as compared to the single threshold method. In other words, the present female classifier model identifies 4× more cancers in female patients as compared to the conventional methods of “any marker high”. See FIG. 7.

Table 9 below provides a normal range for well-validated tumor markers, measurement of a given marker above the normal range would indicate an increased likelihood of developing cancer using conventional methods.

TABLE 9
Female Biomarkers with Well-Validated Performance:

| Biomarker | Normal Range | | Cancers |
|---|---|---|---|
| AFP | <8.3 | ng/ml | Liver cancer, testicular |
| | | | and ovarian cancers |
| CA 19-9 | <35 | U/ml | Pancreatic, colorectal, |
| | | | stomach, liver and bile |
| | | | duct cancer |
| CEA | <4.7 ng/ml | | Colorectal, pancreatic, |
| | (non-smokers) | | gastrointestinal cancers, |
| | <5.6 ng/ml | | lung cancer |
| | (smokers) | | |
| CYFRA 21-1 | <3.3 | ng/ml | Lung, H&N cancer, uterine |
| | | | cancer, esophagus cancer, |
| | | | bladder cancer, mesothelioma, |
| | | | some lymphomas and sarcomas |
| CA 125 | <38 | U/ml | Ovarian and lung cancers |
| CA15-3 | <25 | U/ml | Breast cancer |

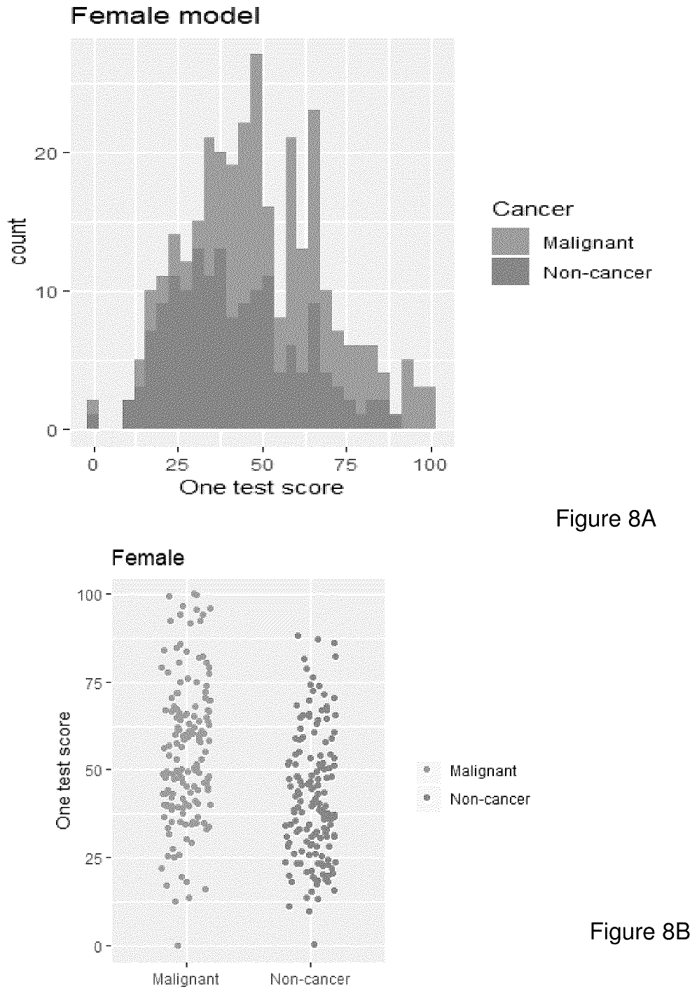

The present female classifier model provides a substantial improvement in diagnostic accuracy over conventional methods, e.g., any marker high methods; an improvement in sensitivity is demonstrated wherein 4× more cancers in females are detected. Moreover, the present female classifier model was able to distinguish cancers from noncancers with 50% sensitivity and 74% specificity. See FIG. 8. In this figure, the cut off between low risk and moderate or high risk was 50, or 0.5. The risk score may be provided from 0 to 1, or 0 to 100, or X out of 100 patients (who have scored (in the population used to develop the algorithm) at or above your score were diagnosed with cancer within one year of have these biomarkers tested). In embodiments, a heterogenous population has a cancer incidence of 1 out 100, wherein any risk score of 1 out of 100 is considered normal risk, or not an increased risk. In further embodiments, a risk score of 2 out of 100, or great, classifies a patient in an increased risk category.

### Example 6: Screening Patients for Likelihood of Developing Cancer and Identifying Patients with an Increased Risk of Developing Cancer when all Measured Biomarkers are in the Normal Range

Provided herein is a method for predicting an increased risk of having or developing cancer, for an asymptomatic patient, wherein a model trained from the cohort in Example 1 is applied to the measured panel of biomarkers and the clinical factors of age and gender to identify those patients with an increased risk of having or developing cancer; the pan-cancer test. In embodiments, this method and present classifier model uses input variables of measured biomarkers that are within a normal clinical range, wherein the pan-cancer classifier model classifies the patient in an increased risk category using input variables of age and the measured values of a panel of biomarkers from the patient when an output of the first classifier model is above a threshold.

In the present example, 4 asymptomatic patients (2 male and 2 female) were screened using the pan-cancer test according to Example 1 and Example 3. In this example, the biomarkers of Table 8 were measured within the normal range, however the present male classifier model classified both patients in an increased risk category using a threshold of a 1% (cancer rate in a heterogenous population). One patient (mp #1) was classified as having an increased risk of having cancer as 5 out of 100 (positive predictive value) and the other (mp #2) was classified as having an increased risk of having cancer as 12 out of 100. Mp #1 was subsequently diagnosed with stage 1 liver cancer and mp #2 was subsequently diagnosed with stage 1 bladder cancer. In both cases, the present male classifier model classified the male patients at high risk, where normally all tumor markers low would not raise concern.

In this example the biomarkers of Table 9 were measured within the normal range, however the present female classifier model classified both patients in an increased risk category using a threshold of a 1% (cancer rate in a heterogenous population). One patient (fp #1) was classified as having an increased risk of having cancer as 2 out of 100 (positive predictive value) and the other (fp #2) was classified as having an increased risk of having cancer as 3 out of 100. Fp #1 was subsequently diagnosed with stage 1B lung cancer and fp #2 was subsequently diagnosed with stage 2B breast cancer. In both cases, the present female classifier model classified the female patients at high risk, where normally all tumor markers low would not raise concern.

1. A method, in a computer-implemented system comprising at least one processor and at least one memory, the at least one memory comprising instructions executed by the at least one processor to cause the at least one processor to implement one or more classifier models to predict an increased risk of having or developing cancer, for an asymptomatic patient, comprising:
a) obtaining measured values of a panel of biomarkers in a sample from the patient, wherein a value of a biomarker corresponds to a level of the biomarker in the sample;
b) obtaining clinical parameters corresponding to the patient including at least age and gender;
c) classifying the patient into a risk category of having or developing cancer using a first classifier model, wherein the first classifier model is generated by a machine learning system using first training data that comprises values of a panel of at least two biomarkers, age, and a diagnostic indicator, for a population of patients; and,
wherein the first classifier model classifies the patient in an increased risk category using input variables of age and the measured values of a panel of biomarkers from the patient when an output of the first classifier model is above a threshold; and,
d) providing a notification to a user for diagnostic testing of the patient when the patient is classified in the increased risk category.

2. The method of claim 1, wherein the first classifier model has a performance of a Receiver Operator Characteristic (ROC) curve with a sensitivity value of at least 0.8 and a specificity value of at least 0.8.

3. The method of claim 1, wherein the first training data comprises values from a panel of at least six biomarkers.

4. The method of claim 1, wherein the input variables comprise measured values from a panel of at least six biomarkers.

5. The method of claim 3, wherein the panel of biomarkers is selected from AFP, CEA, CA125, CA19-9, CA 15-3, CYFRA21-1, PSA and SCC.

6. The method of claim 4, wherein the panel of biomarkers is selected from AFP, CEA, CA125, CA19-9, CA 15-3, CYFRA21-1, PSA and SCC.

7. The method of claim 1, wherein the panel of biomarkers for a male patient is selected from AFP, CEA, CA19-9, CYFRA21-1, PSA and SCC.

8. The method of claim 1, wherein the panel of biomarkers for a female patient is selected from AFP, CEA, CA125, CA19-9, CA 15-3, CYFRA21-1 and SCC.

9. The method of claim 1, wherein the machine learning system further comprises iteratively regenerating the first classifier model by training the first classifier model with new training data to improve the performance of the first classifier model.

10. The method of claim 9, wherein the first classifier model has an improved performance of a Receiver Operator Characteristic (ROC) curve having a sensitivity value of at least 0.85 and a specificity value of at least 0.8.

11. The method of claim 1, wherein the risk category comprises low risk, moderate risk or high risk.

12. (canceled)

13. The method of claim 1, wherein the diagnostic testing is radiographic screening or a tissue biopsy.

14. The method of claim 1, further comprising:
(1) obtaining one or more test results from the diagnostic testing which confirm or deny the presence of cancer in the patient;
(2) incorporating the one or more test results into the first training data for further training of the first classifier model of the machine learning system; and
(3) generating an improved first classifier model by the machine learning system.

15. The method of claim 1, wherein the first classifier model comprises a support vector machine, a decision tree, a random forest, a neural network, a deep learning neural network, or a logistic regression algorithm.

16. The method of claim 1, wherein the cancer is selected from the group consisting of: breast cancer, bile duct cancer, bone cancer, cervical cancer, colon cancer, colorectal cancer, gallbladder cancer, kidney cancer, liver or hepatocellular cancer, lobular carcinoma, lung cancer, melanoma, ovarian cancer, pancreatic cancer, prostate cancer, skin cancer, and testicular cancer.

17. The method of claim 1, wherein the first training data comprises a group of data from a group of patients with no cancer diagnosis three or more months after providing a sample.

18. The method of claim 1, wherein the first training data comprises a group of data from a group of patients with a cancer diagnosis three or more months after providing a sample.

19. The method of claim 1, wherein the threshold is a probability value of 0.5.

20. The method of claim 1, wherein the first training data comprises a greater number of patients without cancer than with cancer, and further comprising:
reprocessing the first training data by using a stratified sampling technique to improve selection of negative samples.

21. The method of claim 1, wherein patients classified into the increased risk category by the first classifier model are further classified using a second classifier model, wherein the second classifier model is generated by the machine learning system using second training data that comprises values of a panel of at least two biomarkers and a diagnostic indicator from a population of patients, wherein the second classifier model predicts at least one most likely organ system malignancy for that patient by assigning a class membership corresponding to the most likely organ system malignancy, using input variables of the measured values of the panel of biomarkers from the patient.

22-39. (canceled)
